(19) United States
(12) Patent Application Publication
(10) Pub. No.: US 2026/0102057 A1
(43) Pub. Date: Apr. 16, 2026

(54) ACTUATABLE URETEROSCOPE DRIVER

(71) Applicant: Auris Health, Inc., Santa Clara, CA (US)

(72) Inventor: David COWENS, Santa Clara, CA (US)

(73) Assignee: Auris Health, Inc., Santa Clara, CA (US)

(21) Appl. No.: 19/335,561

(22) Filed: Sep. 22, 2025

Related U.S. Application Data
(60) Provisional application No. 63/707,666, filed on Oct. 15, 2024.
(60) Provisional application No. 63/846,376, filed on Jul. 18, 2025.
(60) Provisional application No. 63/875,491, filed on Sep. 4, 2025.

(51) Int. Cl. A61B 1/00; A61B 1/307; A61B 34/30; A61B 34/37; A61M 25/01

(52) U.S. Cl. CPC A61B 1/0016; A61B 1/00149; A61B 1/307; A61B 34/37; A61B 2034/301; A61B 2034/303; A61M 25/0113

(57) ABSTRACT

A scope driver is disclosed that includes a housing defining a channel sized to receive a scope, a first rotary input driver rotatably mounted to the housing, a drive wheel arranged within the housing and operatively coupled to the first rotary input driver such that rotation of the first rotary input driver correspondingly rotates the drive wheel, a second rotary input driver rotatably mounted to the housing, and an idler wheel mounted to a swing arm within the housing and operatively coupled to the second rotary input driver such that actuation of the second rotary input driver correspondingly reciprocates the idler wheel toward or away from the drive wheel. Moving the idler wheel toward the drive wheel engages the scope between the drive and idler wheels and moving the idler wheel away from the drive wheel disengages the scope from at least one of the drive and idler wheels.

### CROSS-REFERENCE TO RELATED APPLICATIONS

The present application claims priority under 35 U.S.C. § 119(e) to U.S. Provisional Patent Application Ser. No. 63/707,666, titled URETEROSCOPE DRIVER, filed Oct. 15, 2024, U.S. Provisional Patent Application Ser. No. 63/846,376, titled ACTUATABLE URETEROSCOPE DRIVER, filed Jul. 18, 2025, and U.S. Provisional Patent Application Ser. No. 63/875,491, titled ACTUATABLE URETEROSCOPE DRIVER, filed Sep. 4, 2025, the disclosures of which are hereby incorporated by reference in their entirety.

### BACKGROUND

Kidney stone disease, also known as urolithiasis, is a medical condition that involves the formation in the urinary tract of a solid piece of material, referred to as “kidney stones,” “urinary stones,” “renal calculi,” “renal lithiasis,” or “nephrolithiasis.” Urinary stones may be formed and/or found in the kidneys, the ureters, and the bladder (referred to as “bladder stones”). Such urinary stones can form as a result of mineral concentration in urinary fluid and can cause significant abdominal pain once such stones reach a size sufficient to impede urine flow through the ureter or urethra. Urinary stones may be formed from calcium, magnesium, ammonia, ur acid, cysteine, and/or other compounds or combinations thereof.

Generally, there are several methods for treating patients with kidney stones, including observation, medical treatments (such as expulsion therapy), non-invasive treatments (such as extracorporeal shock wave lithotripsy (ESWL)), and surgical treatments (such as ureteroscopy and percutaneous nephrolithotomy (“PCNL”)). In surgical approaches (e.g., ureteroscopy and PCNL), the physician gains access to the object to be removed (e.g., a kidney stone), the object is broken into smaller pieces or fragments, and the relatively small stone fragments/particulates are mechanically extracted from the kidney.

To remove urinary stones from the bladder and ureter, surgeons may insert a ureteroscope into the urinary tract through the urethra. Typically, a ureteroscope includes a shaft with a scope/camera at the distal end thereof that is configured to enable visualization of the urinary tract. The ureteroscope can also include a lithotripsy device to capture or break apart urinary stones. During a ureteroscopy procedure, one physician/technician may control the position of the ureteroscope, while another physician/technician may control the lithotripsy device(s).

Robotic devices and/or systems can be employed in connection with various medical procedures, such as kidney stone removal procedures, wherein robotic tools can enable a physician/urologist to perform endoscopic (e.g., ureteroscopy) target access, percutaneous access/treatment, or other aspects of a medical procedure. These robotic systems can control the position of the ureteroscope in the urinary track. The shafts of the ureteroscope tend to be wet and slippery, which can make the ureteroscope drive system slip as it drives the ureteroscope within the patient. This slippage may cause uncertainty regarding the position of the ureteroscope within the patient.

Accordingly, systems and methods for driving a ureteroscope within a patient that experience a lower degree (or no) slippage, thereby increasing the confidence of the position of the ureteroscope within a patient, are desired.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 illustrates an example medical system including one or more basketing components in accordance with one or more embodiments.

FIG. 2 illustrates medical system components that may be implemented in the medical system of FIG. 1 in accordance with one or more embodiments.

FIGS. 3A-3E illustrate an example basketing system in various configurations in accordance with one or more embodiments.

FIG. 4 is an example scope driver coupled to an arm of a robotic surgical system via a sterile barrier adapter, in accordance with at least one aspect of the present disclosure.

FIG. 5 is an isometric view of a drive interface of the arm of FIG. 4, in accordance with at least one aspect of the present disclosure.

FIG. 6 is an isometric view of the sterile barrier adapter of FIG. 4, in accordance with at least one aspect of the present disclosure.

FIG. 7 is a top-down view of FIG. 4 with a cover of the scope driver removed, in accordance with at least one aspect of the present disclosure.

FIG. 8A is an underside view of the cover of the scope driver, in accordance with at least one aspect of the present disclosure.

FIG. 8B is a top-down view of a base of the scope driver of FIG. 4, in accordance with at least one aspect of the present disclosure.

FIG. 9A is an isometric view of an example drive assembly of the scope driver of FIG. 4, in accordance with at least one aspect of the present disclosure.

FIG. 9B is another isometric view of the drive assembly of FIG. 9A, in accordance with at least one aspect of the present disclosure.

FIG. 9C is a bottom view of the drive assembly of FIG. 9A, in accordance with at least one aspect of the present disclosure.

FIG. 9D is an isometric view of a second rotary input driver of the drive assembly of FIG. 9A, in accordance with at least one aspect of the present disclosure.

FIGS. 9E and 9F are isometric top and bottom views, respectively, of a lower swing arm portion of the drive assembly of FIG. 9A, in accordance with at least one aspect of the present disclosure.

FIGS. 9G and 9H are isometric top and bottom views, respectively, of an upper swing arm portion of the drive assembly of FIG. 9A, in accordance with at least one aspect of the present disclosure.

FIG. 10A is an isometric view of a scope driver from a top right perspective, in accordance with at least one aspect of the present disclosure.

FIG. 10B is an isometric view of the scope driver of FIG. 10A from a bottom left perspective, in accordance with at least one aspect of the present disclosure.

FIG. 10C is an isometric view of a bailout and a partial view of a drive system of the scope driver of FIG. 10A, in accordance with at least one aspect of the present disclosure.

FIG. 11A is an isometric view of a scope driver from a top right perspective, in accordance with at least one aspect of the present disclosure.

FIG. 11B is an isometric view of a bailout and a partial view of a drive system of the scope driver of FIG. 11A, in accordance with at least one aspect of the present disclosure.

### DETAILED DESCRIPTION

The present disclosure relates to surgical systems and, more particularly, to drive systems for driving a ureteroscope within a patient.

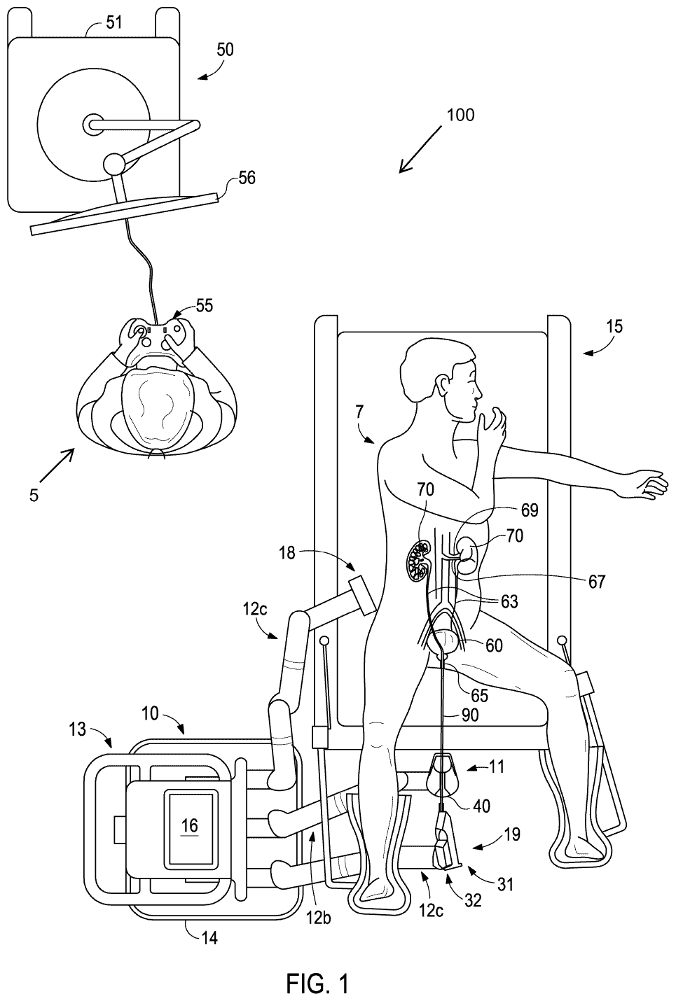

FIG. 1 illustrates an example medical system 100 for performing various medical procedures and that may incorporate aspects of the present disclosure. The medical system 100 may be used for, for example, endoscopic (e.g., ureteroscopic) procedures. As referenced and described above, certain ureteroscopic procedures involve the treatment/removal of kidney stones. In some implementations, kidney stone treatment can benefit from the assistance of certain robotic technologies/devices, such as may be similar to those shown in FIG. 1 and described in detail below. Robotic medical solutions can provide relatively higher precision, superior control, and/or superior hand-eye coordination with respect to certain instruments compared to strictly-manual procedures. For example, robotic-assisted ureteroscopic access to the kidney in accordance with some procedures can advantageously enable a urologist to perform both endoscope control and basketing control.

Although the system 100 of FIG. 1 is presented in the context of a ureteroscopic procedure, it should be understood that the principles disclosed herein may be implemented in any type of endoscopic and/or percutaneous procedure. Furthermore, several of the examples described herein relate to object removal procedures involving the removal of kidney stones from a kidney. The present disclosure, however, is not limited only to kidney stone removal. For example, the following description is also applicable to other surgical or medical operations or medical procedures concerned with the removal of objects from a patient, including any object that can be removed from a treatment site or patient cavity (e.g., the esophagus, ureter, intestine, eye, etc.) via percutaneous and/or endoscopic access, such as, for example, gallbladder stone removal, lung (pulmonary/transthoracic) tumor biopsy, or cataract removal.

The medical system 100 includes a robotic system 10 (e.g., mobile robotic cart) configured to engage with and/or control a medical instrument 40 (e.g., ureteroscope) to perform a direct-entry procedure on a patient 7. The term “direct-entry” is used herein according to its broad and ordinary meaning and may refer to any entry of instrumentation through a natural or artificial opening in a patient's body. For example, with reference to FIG. 1, the direct entry of the scope 40 into the urinary tract of the patient 7 may be made via the urethra 65.

It should be understood that the direct-entry instrument 40 may be any type of medical instrument, including an endoscope (such as a ureteroscope), catheter (such as a steerable or non-steerable catheter), nephroscopes, laparoscope, or other type of medical instrument. Embodiments of the present disclosure relating to basketing solutions implemented in connection with ureteroscopic procedures for removal of kidney stones through a ureteral access sheath (e.g., the ureteral access sheath 90) are also applicable to solutions for removal of objects through percutaneous access, such as through a percutaneous access sheath. For example, instrument(s) may access the kidney percutaneously through, for example, a percutaneous access sheath to capture and remove kidney stones; instruments used to capture such stones may become stuck on the internal renal anatomy and/or the percutaneous access sheath (e.g., at an opening of the percutaneous access sheath). The term “percutaneous access” is used herein according to its broad and ordinary meaning and refers to entry, such as by puncture and/or minor incision, of instrumentation through the skin of a patient and any other body layers necessary to reach a target anatomical location associated with a procedure (e.g., the calyx network of the kidney 70).

The medical system 100 includes a control system 50 configured to interface with the robotic system 10, provide information regarding the procedure, and/or perform a variety of other operations. For example, the control system 50 includes one or more display(s) 56 configured to present certain information to assist the physician 5 and/or other technician(s) or individual(s). The medical system 100 can include a table 15 configured to hold the patient 7. The system 100 may further include an electromagnetic (EM) field generator 18, which may be held by one or more of the robotic arms 12 of the robotic system 10 or may be a stand-alone device. Although the various robotic arms 12 are shown in various positions and coupled to various instrumentation, it should be understood that such configurations are shown for convenience and illustration purposes, and such robotic arms may have different configurations over time and/or at different points during a medical procedure. Furthermore, the robotic arms 12 may be coupled to different instruments than shown in FIG. 1, and in some cases or periods of time, one or more of the arms 12 may not be utilized or coupled to a medical instrument (e.g., instrument manipulator/coupling).

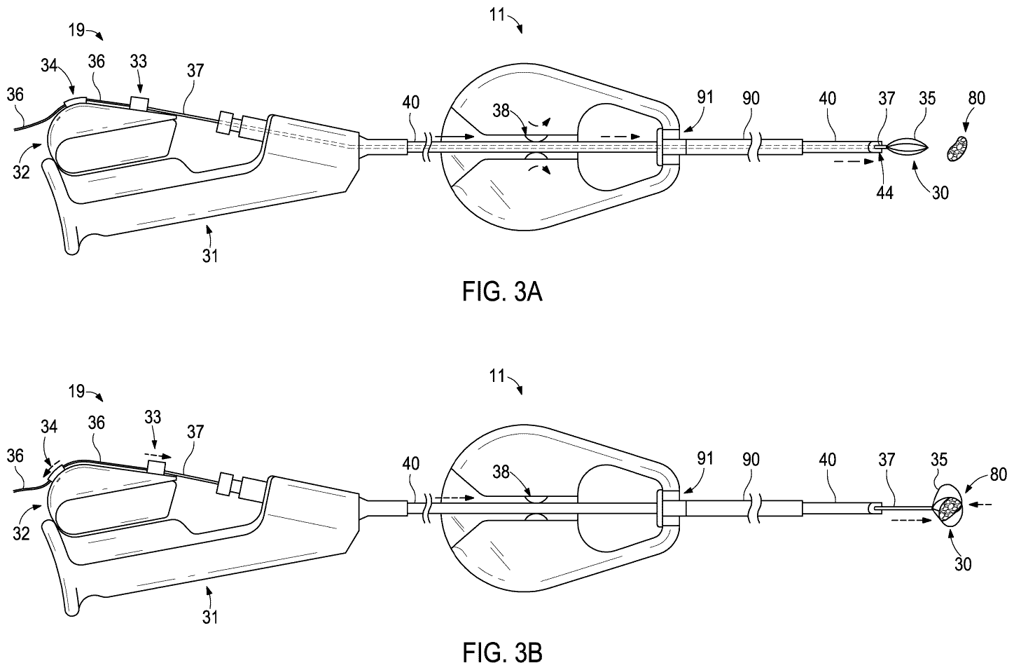

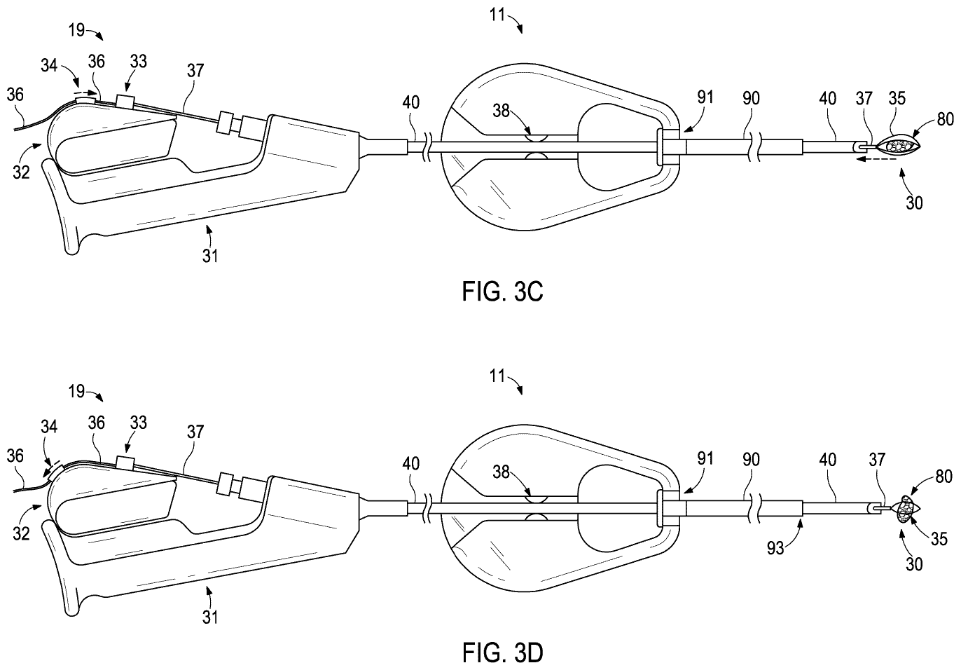

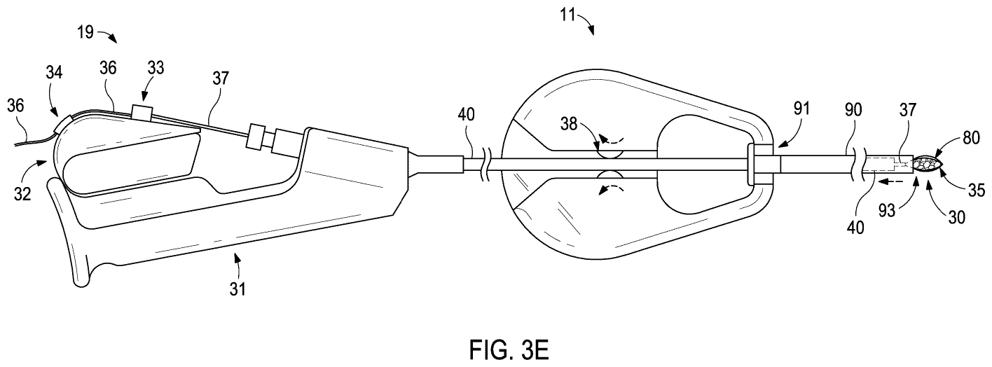

In an example use case, with reference to FIG. 3A, if the patient 7 has a kidney stone 80 located in the kidney 70, the physician executes a procedure to remove the stone 80 through the urinary tract (63, 60, 65). In some embodiments, the physician 5 can interact with the control system 50 and/or the robotic system 10 to cause/control the robotic system 10 to advance and navigate the medical instrument 40 (e.g., a scope) from the urethra 65, through the bladder 60, up the ureter 63, and into the kidney 70 where the stone 80 is located. The physician 5 can further interact with the control system 50 and/or the robotic system 10 to cause/control the advancement of a basketing device 30 through a working channel of the instrument 40, wherein the basketing device 30 is configured to facilitate capture and removal of a kidney stone. The control system 50 can provide information via the display(s) 56 that is associated with the medical instrument 40, such as real-time endoscopic images captured therewith, and/or other instruments of the system 100, to assist the physician 5 in navigating/controlling such instrumentation.

The medical instrument 40 (e.g., scope, directly-entry instrument, etc.) can be advanced into the kidney 70 through the urinary tract. Specifically, a ureteral access sheath 90 may be disposed within the urinary tract and advanced to an area near the kidney 70. The medical instrument 40 may be passed through the ureteral access sheath 90 to gain access to the internal anatomy of the kidney 70, as shown. Once at the site of the kidney stone 80, the medical instrument 40 can be used to channel/direct the basketing device 30 to the target location. Once the stone 80 has been captured in the distal basket portion 35 of the basketing device 30, the utilized ureteral access path may be used to extract the kidney stone 80 from the patient 7.

The various scope-type instruments disclosed herein, such as the scope 40 of the system 100, can be configured to navigate within the human anatomy, such as within a natural orifice or lumen of the human anatomy. The terms “scope” and “endoscope” are used herein according to their broad and ordinary meanings, and may refer to any type of elongate medical instrument having image generating, viewing, and/or capturing functionality and configured to be introduced into any type of organ, cavity, lumen, chamber, or space of a body. A scope can include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise a rigid or flexible tube, and may be dimensioned to be passed within an outer sheath, catheter, introducer, or other lumen-type device, or may be used without such devices.

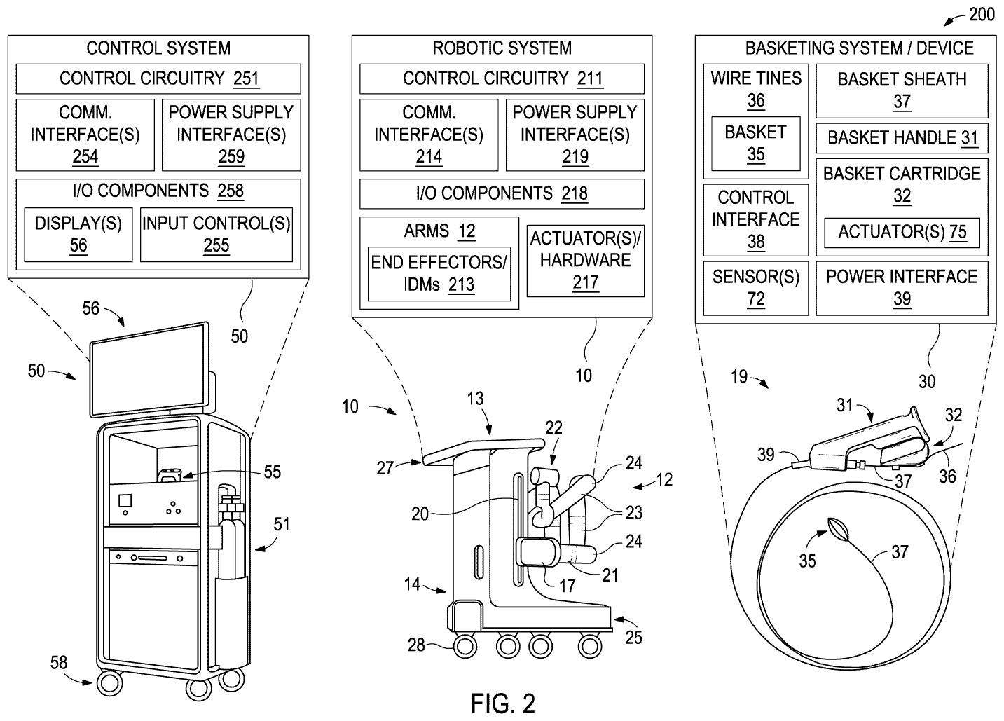

With reference again to FIG. 1 and FIG. 2, which shows an example embodiment of the control system 50 of FIG. 1 in accordance with one or more embodiments of the present disclosure, the control system 50 can be configured to provide various functionality to assist in performing a medical procedure. In some embodiments, the control system 50 can be coupled to the robotic system 10 and operate in cooperation therewith to perform a medical procedure on the patient 7. For example, the control system 50 can communicate with the robotic system 10 via a wireless or wired connection (e.g., to control the robotic system 10). Further, in some embodiments, the control system 50 can communicate with the robotic system 10 to receive position data therefrom relating to the position of the distal end of the scope 40, access sheath 90, or basketing device 30. Such positional data relating to the position of the scope 40, access sheath 90, or basketing device 30 may be derived using one or more electromagnetic sensors associated with the respective components. Moreover, in some embodiments, the control system 50 can communicate with the table 15 to position the table 15 in a particular orientation or otherwise control the table 15. In some embodiments, the control system 50 can communicate with the EM field generator 18 to control generation of an EM field in an area around the patient 7.

FIG. 2 further shows an example of the robotic system 10 of FIG. 1 in accordance with one or more embodiments of the present disclosure. The robotic system 10 can be configured to at least partly facilitate execution of a medical procedure. The robotic system 10 can be arranged in a variety of ways depending on the particular procedure. The robotic system 10 can include one or more robotic arms 12 configured to engage with and/or control, for example, the scope 40 and/or the basketing system 30 to perform one or more aspects of a procedure. As shown, each robotic arm 12 can include multiple arm segments 23 coupled to joints 24, which can provide multiple degrees of movement/freedom. In the example of FIG. 1, the robotic system 10 is positioned proximate to the patient's legs and the robotic arms 12 are actuated to engage with and position the scope 40 for access into an access opening, such as the urethra 65 of the patient 7. When the robotic system 10 is properly positioned, the scope 40 can be inserted into the patient 7 robotically using the robotic arms 12, manually by the physician 5, or a combination thereof. A scope-driver instrument coupling or “scope driver” 11 (i.e., instrument device manipulator (IDM)) can be attached to the distal portion of one of the arms 12b to facilitate robotic control/advancement of the scope 32. Another 12c of the arms has associated therewith an instrument coupling/manipulator 19 configured to facilitate advancement and operation of the basketing device 30. The scope 40 includes one or more working channels through which additional tools, such as lithotripters, basketing devices, forceps, etc., can be introduced into the treatment site.

The robotic system 10 can be coupled to any component of the medical system 100, such as to the control system 50, the table 15, the EM field generator 18, the scope 40, the basketing system 30, and/or any type of percutaneous-access instrument (e.g., needle, catheter, nephroscope, etc.). In some embodiments, the robotic system 10 is communicatively coupled to the control system 50. For example, the robotic system 10 may be configured to receive control signals from the control system 50 to perform certain operations, such as to position one or more of the robotic arms 12 in a particular manner, manipulate the scope 40, manipulate the basketing system 30, and so on. In response, the robotic system 10 can control, using certain control circuitry 211, actuators 217, and/or other components of the robotic system 10, a component of the robotic system 10 to perform the operations. In some embodiments, the robotic system 10 and/or control system 50 is/are configured to receive images and/or image data from the scope 40 representing internal anatomy of the patient 7, namely the urinary system with respect to the particular depiction of FIG. 1, and/or display images based thereon.

With reference to FIG. 2, the robotic system 10 generally includes an elongated support structure 14 (also referred to as a “column”), a robotic system base 25, and a console 13 at the top of the column 14. The column 14 includes one or more arm supports 17 (also referred to as a “carriage”) for supporting the deployment of the one or more robotic arms 12 (three shown in FIG. 2). The arm support 17 includes individually-configurable arm mounts that rotate along a perpendicular axis to adjust the base of the robotic arms 12 for desired positioning relative to the patient.

The arm support 17 is configured to vertically translate along the column 14. In some embodiments, the arm support 17 can be connected to the column 14 through slots 20 that are positioned on opposite sides of the column 14 to guide the vertical translation of the arm support 17. The slot 20 contains a vertical translation interface to position and hold the arm support 17 at various vertical heights relative to the robotic system base 25. Vertical translation of the arm support 17 allows the robotic system 10 to adjust the reach of the robotic arms 12 to meet a variety of table heights, patient sizes, and physician preferences. Similarly, the individually-configurable arm mounts on the arm support 17 can allow the robotic arm base 21 of robotic arms 12 to be angled in a variety of configurations.

The robotic arms 12 generally include robotic arm bases 21 and end effectors 22, separated by a series of linking arm segments 23 that are connected by a series of joints 24, each joint including one or more independent actuators 217. Each actuator 217 comprises an independently-controllable motor. Each independently-controllable joint 24 can provide or represent an independent degree of freedom available to the robotic arm. In some embodiments, each of the arms 12 has seven joints, and thus provides seven degrees of freedom, including “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The robotic system base 25 balances the weight of the column 14, arm support 17, and arms 12 over the floor. Accordingly, the robotic system base 25 houses certain relatively heavier components, such as electronics, motors, power supply, as well as components that selectively enable movement or immobilize the robotic system. The robotic system base 25 can include wheel-shaped casters 28 that allow for the robotic system to easily move around the operating room prior to a procedure. After reaching the appropriate position, the casters 28 can be immobilized using wheel locks to hold the robotic system 10 in place during the procedure.

Positioned at the upper end of column 14, the console 13 can provide both a user interface for receiving user input and a display screen 16 (or a dual-purpose device such as, for example, a touchscreen) to provide the physician/user with both pre-operative and intra-operative data. Potential pre-operative data on the console/display 16 or display 56 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 13 may be positioned and tilted to allow a physician to access the console from the side of the column 14 opposite arm support 17. From this position, the physician may view the console 13, robotic arms 12, and patient while operating the console 13 from behind the robotic system 10. As shown, the console 13 can also include a handle 27 to assist with maneuvering and stabilizing robotic system 10.

The end effector 213 of each of the robotic arms 12 includes, or is configured to have coupled thereto, an instrument device manipulator (IDM), which is attached using a mechanism changer interface (MCI). In some embodiments, the IDM can be removed and replaced with a different type of IDM, for example, a first type 11 of IDM manipulates an endoscope, while a second type 19 of IDM manipulates a basketing device. Another type of IDM is configured to hold an electromagnetic field generator 18. An MCI can provide power and control interfaces. For example, the interfaces can include connectors to transfer pneumatic pressure, electrical power, electrical signals, and/or optical signals from the robotic arm 12 to the IDM. The IDMs 213 may be configured to manipulate medical instruments (e.g., surgical tools/instruments), such as the scope 40, using techniques including, for example, direct drives, harmonic drives, geared drives, belts and pulleys, magnetic drives, and the like. In some embodiments, the medical device manipulators 213 can be attached to respective ones of the robotic arms 212, wherein the robotic arms 212 are configured to insert or retract the respective coupled medical instruments into or out of the treatment site.

As referenced above, the system 100 can include certain control circuitry configured to perform certain of the functionality described herein, including the control circuitry 211 of the robotic system 10 and the control circuitry 251 of the control system 50. That is, the control circuitry of the system 100 may be part of the robotic system 10, the control system 50, or some combination thereof. Therefore, any reference herein to control circuitry may refer to circuitry embodied in a robotic system, a control system, or any other component of a medical system, such as the medical system 100 shown in FIG. 1. The term “control circuitry” is used herein according to its broad and ordinary meaning, and refers to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 211/251 may be entirely locally maintained/disposed or may be remotely located at least in part (e.g., communicatively coupled indirectly via a local area network and/or a wide area network). Any of the control circuitry 211, 251 may be configured to perform any aspect(s) of the various processes disclosed herein.

With respect to the robotic system 10, at least a portion of the control circuitry 211 can be integrated with the base 25, column 14, and/or console 13 of the robotic system 10, and/or another system communicatively coupled to the robotic system 10. With respect to the control system 50, at least a portion of the control circuitry 251 may be integrated with the console base 51 and/or display unit 56 of the control system 50.

With further reference to FIG. 2, the control system 50 can include various I/O components 258 configured to assist the physician 5 or others in performing a medical procedure. For example, the input/output (I/O) components 258 can be configured to allow for user input to control/navigate the scope 40 and/or basketing system within the patient 7. In some embodiments, for example, the physician 5 can provide input to the control system 50 and/or robotic system 10, wherein in response to such input, control signals can be sent to the robotic system 10 to manipulate the scope 40 and/or catheter basketing system 30. The control system 50 can include one or more display devices 56 to provide various information regarding a procedure. For example, the display(s) 56 can provide information regarding the scope 40 and/or basketing system 30. For example, the control system 50 can receive real-time images that are captured by the scope 40 and display the real-time images via the display(s) 56. Additionally, or alternatively, the control system 50 can receive signals (e.g., analog, digital, electrical, acoustic/sonic, pneumatic, tactile, hydraulic, etc.) from a medical monitor and/or a sensor associated with the patient 7, and the display(s) 56 can present information regarding the health or environment of the patient 7. Such information can include information that is displayed via a medical monitor including, for example, information relating to heart rate (e.g., ECG, HRV, etc.), blood pressure/rate, muscle bio-signals (e.g., EMG), body temperature, blood oxygen saturation (e.g., SpO₂), CO₂, brainwaves (e.g., EEG), environmental and/or local or core body temperature, and so on.

To facilitate the functionality of the control system 50, the control system can include various components or “subsystems”. For example, the control system 50 can include the control electronics/circuitry 251, as well as one or more power supplies/supply interfaces 259, pneumatic devices, optical sources, actuators, data storage devices, and/or communication interfaces 254. In some embodiments, the control system 50 is movable, while in other embodiments, the control system 50 is a substantially stationary system.

Referring again to FIG. 1, the medical system 100 can provide a variety of benefits, such as providing guidance to assist a physician in performing a procedure (e.g., instrument tracking, instrument alignment information, etc.), enabling a physician to perform a procedure from an ergonomic position without the need for awkward arm motions and/or positions, enabling a single physician to perform a procedure with one or more medical instruments, avoiding radiation exposure (e.g., associated with fluoroscopy techniques), enabling a procedure to be performed in a single operative setting, providing continuous suction to remove an object more efficiently (e.g., to remove a kidney stone), and so on. For example, the medical system 100 can provide guidance information to assist a physician in using various medical instruments to access a target anatomical feature while minimizing bleeding and/or damage to anatomy (e.g., critical organs, blood vessels, etc.). Further, the medical system 100 can provide non-radiation-based navigational and/or localization techniques to reduce physician and patient exposure to radiation and/or reduce the amount of equipment in the operating room. Moreover, the medical system 100 can provide functionality that is distributed between the control system 50 and the robotic system 10, which may be independently movable. Such distribution of functionality and/or mobility can enable the control system 50 and/or the robotic system 10 to be placed at locations that are optimal for a particular medical procedure, which can maximize working area around the patient 7 and/or provide an optimized location for the physician 5 to perform a procedure.

The various components of the system 100 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, personal area networks (PANs), body area network (BANs), etc. For example, the various communication interfaces of the systems of FIG. 2 can be configured to communicate with one or more device/sensors/systems, such as over a wireless and/or wired network connection. In some embodiments, the various communication interfaces can implement a wireless technology such as Bluetooth, Wi-Fi, near field communication (NFC), or the like. Furthermore, in some embodiments, the various components of the system 100 can be connected for data communication, fluid exchange, power exchange, and so on via one or more support cables, tubes, or the like.

The control system 50, basking system 30, and/or robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore. Such user controls are communicatively and/or physically coupled to respective control circuitry.

In some embodiments, a user can manually manipulate a robotic arm 12 of the robotic system 10 without using electronic user controls. For example, during setup in a surgical operating room, a user may move the robotic arms 12 and/or any other medical instruments to provide desired access to a patient. The robotic system 10 may rely on force feedback and inertia control from the user to determine appropriate configuration of the robotic arms 12 and associated instrumentation.

The basketing system 30 includes various hardware and control components. For example, as shown in FIG. 2, the processing system 30 can include a basket 35 formed of one or more wire tines 36, such as four wire tines disposed within a basketing sheath 37 over a length thereof, wherein the tines project from a distal end of the sheath 37 to form the basket form 35. The tines 36 further extend from the proximal end of the sheath 37. The tines 36 may be configured to be slidable within the basketing sheath 37, subject to some amount of frictional resistance. The tines 36 and the sheath 37 can be coupled to respective actuators 75 of a basket cartridge component 32. The relationship between the actuators 75 of the basket cartridge 32 and the tines 36 and sheath 37 is described in detail below with respect to FIGS. 3A-3E. The basket cartridge 32 may be physically and/or communicatively coupled to a handle portion/component 31 of the basketing system 30. The handle component 31 can be configured to be used to assist in basketing control either manually or through robotic control.

The basketing system 30 can be powered through a power interface 39 and/or controlled through a control interface 38, each or both of which may interface with a robotic arm/component of the robotic system 10. The basketing system 30 further includes one or more sensors 72, such as pressure and/or other force-reading sensors, which are configured to generate signals indicating forces experienced at/by one or more of the actuators 75 and/or other couplings of the basketing system 30. In some embodiments, the sensor(s) 72 include one or more sensors configured to directly measure forces are at or near the basket portion 35 of the tines 36. For example, a force sensor on the tip of the basket 35 and/or at a tip of an access sheath through which the basketing device 30 accesses the target anatomy can be used to directly detect forces on the basket 35 that result from the basket 35 becoming stuck on anatomy or on an opening at an end of the access sheath.

FIGS. 3A-3D illustrate an example basketing control system in various configurations during example operation. In connection with the various embodiments of the present disclosure, basketing is performed at least in part using one or more robotic instrument device manipulators (IDMs), such as the scope driver IDM 11 and a basketing IDM 19.

In some embodiments, the basketing IDM 19 includes a handle component or “handle” 31 and a basketing cartridge component or “basketing cartridge” 32. The handle 31 is coupled to a proximal end of the scope 40, and includes a channel through which a basketing device sheath 37 is able to enter the scope 40, wherein the basketing sheath 37 (along with basketing tines disposed there) is disposed at least partially within a working channel 44 of the scope 40. The scope 40 and the basketing device 30 are in a relatively fixed position at the handle component 31, wherein the relative position between the scope 40 and the basket 30 may be changed through actuation of one or more of the actuators 33, 34 of the basketing cartridge 32. Actuation of the basketing sheath actuator 33 can cause insertion and retraction of the basketing device 30 relative to the scope 40.

The scope 40 is advanced from the distal end of the access sheath 90 by driving one or more actuators 38 associated with the scope-driver IDM 11. Such actuators 38 can comprise wheel-type actuators used to advance and retract the scope 40. During a kidney stone removal procedure, the actuators 38 are utilized to retract the scope 40 following successful capture of a kidney stone 80 in the basket 35, as shown in FIG. 3E. In some embodiments, the access sheath 90 is coupled to the scope-driver IDM 11 in a fixed manner using a sheath coupling component 91.

The basketing cartridge 32 can include a plurality of actuators 33, 34, such as sliding, carriage-type actuators. Specifically, the cartridge 32 may include a first actuator 33 that is fixed to the sheath component 37 of the basketing device 30, as well as a second actuator 34 that is fixed to the wires/tines 36 of the basketing device 30, wherein the tines 36 may pass through and/or otherwise be disposed at least partially within the basketing sheath 37. In some embodiments, the basket 35 is formed of the tines 36 at a distal portion thereof that projects out of the distal end of the basketing sheath 37.

By sliding the sheath actuator 33, the basketing device 30 is projected from the distal end of the scope 40. For example, as shown in FIG. 3B, the actuator 30 is slid (moved) forward to produce a corresponding forward advancement of the basketing device 30 and basket 35. The basket actuator 34 can be used to open the basket 35 by pulling the tines proximally, thereby causing the distal end of the basket 35 to be pulled toward the distal opening of the sheath 37, thereby resulting in outward bowing/expansion of the basket portion 35 of the tines. With the tines 36 in the expanded/open position shown in FIG. 3B, the basket 35 may be placed around a stone/object 80 to thereby capture the stone/object 80 within the basket tines 36.

As shown in FIG. 3C, the basket 35 is collapsed around the stone 80 to thereby capture the stone therein. For example, the basket tine actuator 34 may be advanced some amount relative to the sheath actuator 33 to thereby push the basket tines 36 farther out of the sheath 37 at a distal end thereof, thereby elongating/extending the basket 35 and bringing the tines closer to an axis of the basketing device 30. The sheath actuator 33 may be drawn proximally to bring the basketing device 30 back closer to the distal end of the scope 40. For example, it may be desirable to have the basket 35 disposed proximal/adjacent to the distal end of the scope 40 during retraction of the scope and basketing device 30.

In some implementations, the basket 35 may be further cinched/collapsed around the stone 80 by drawing the tines 36 further proximally, to thereby draw the tines 36 farther into the sheath 37 and reduce the length of the tines that project distally from the distal end of the basketing sheath 37. FIG. 3D shows the basket 35 with a reduced size resulting from proximally moving the actuator 34 relative to the actuator 33.

Once the stone 80 is captured and the basket 35 is brought to the desired position near the distal end of the scope 40, the actuator(s) 38 are engaged to retract the scope 40 through the opening 93 of the access sheath 90, and further through the access sheath 90, as shown in FIG. 3E. In some embodiments, the basketing device 30 is retracted along with the scope 40 as the scope 40 is proximally drawn. For example, frictional forces between the basketing sheath 37 and the working channel 44 of the scope 40 can be such as to cause the basketing device 30 to accompany the scope 40 when the latter is retracted/moved.

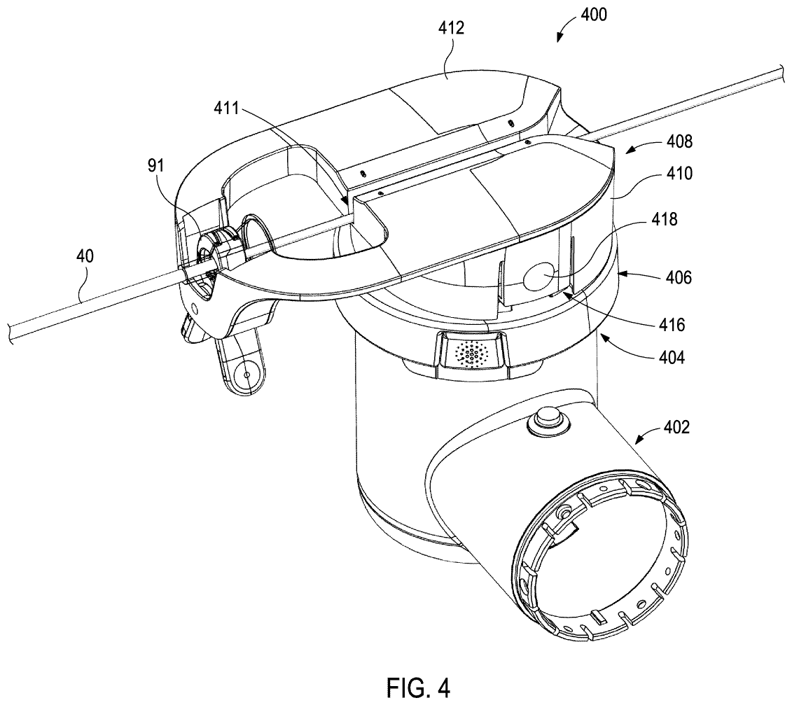

FIG. 4 is an isometric view of an example scope driver 400 coupled to an arm 402 of a robotic system, according to at least one aspect of the present disclosure. The scope driver 400 and the arm 402 may be similar in some respects to the scope driver 11 and arm 12b, respectively, of FIG. 1, and therefore may be best understood with reference thereto, where like numerals will correspond to like components not described again in detail. It should be noted that only the end effector of the arm 402, similar to end effector 22 (FIG. 2), is shown with the remainder of the arm 402 omitted. However, it should be understood that the omitted portions of the arm 402 extend to a robotic system, like robotic system 10, such as via a series of linking arm segments 23 (FIG. 2) that are connected by a series of joints 24 (FIG. 2).

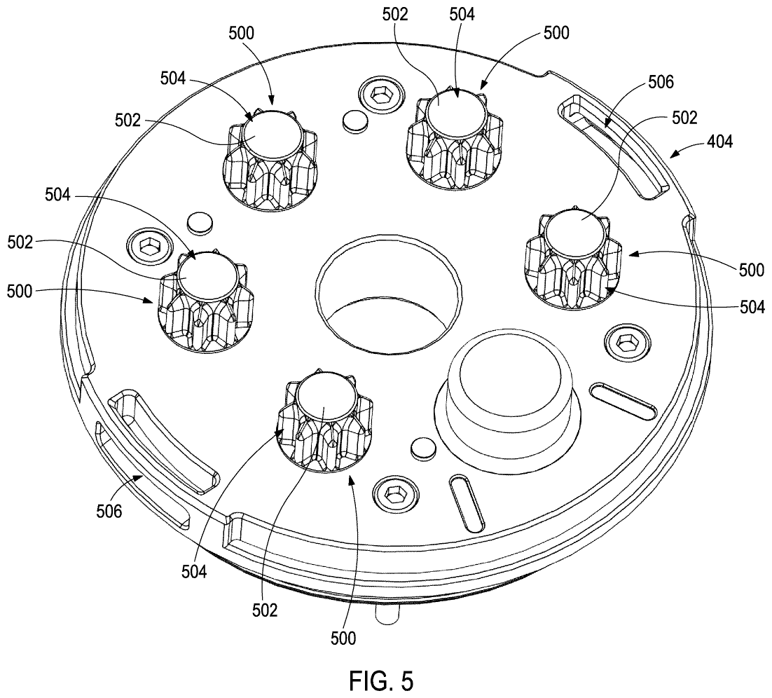

The arm 402 includes a drive interface 404. Referring to FIG. 5, the drive interface includes a plurality of rotary output drivers 500 for driving various functions of the scope driver 400, as explained in more detail herein below. Each rotary output driver 500 may be drivable (rotatable) by a corresponding motor (not shown) energized by the robotic system (e.g., robotic system 10). Each motor 500 may be selectively energized based on a user providing an input to the robotic system, such as via the controls 55 of the control system 50. As illustrated, each rotary output driver 500 includes a spline shaft 502 that defines, or otherwise provides, a plurality of teeth 504. The drive interface 404 may further define a plurality of recesses 506 (e.g., two recesses 506), described in more detail below. While five rotary output drivers 500 are shown and described, the drive interface 404 can include less (e.g., one, two, three, or four) or more (e.g., six, seven, or eight) than five rotary output drivers 500.

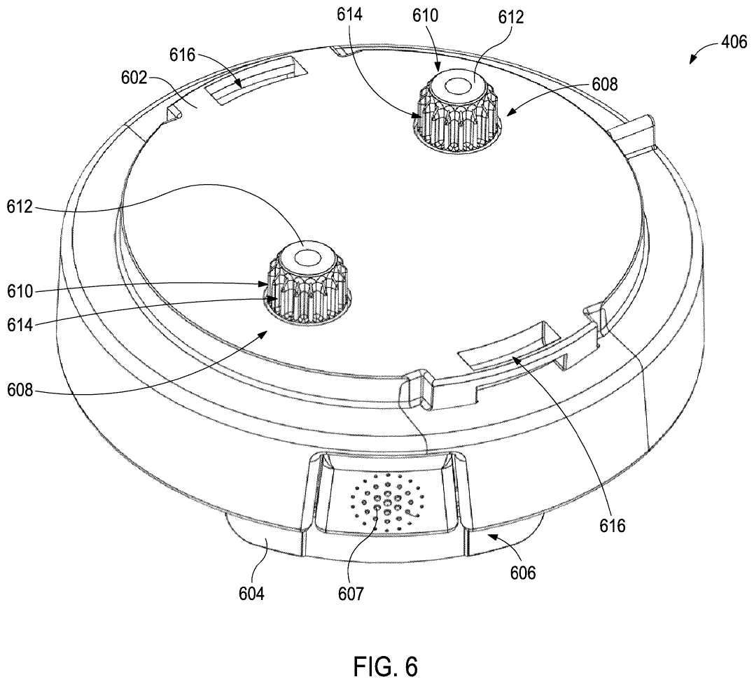

With reference now to FIGS. 4 and 6, the scope driver 400 may be coupled to the drive interface 404 via a sterile barrier adapter 406. The sterile barrier adapter 406 can include a first or “upper” body portion 602 and a second or “lower” body portion 604 removably coupled to the upper body portion 602. The lower body portion 604 may define a plurality of apertures (not shown) for receiving the rotary output drivers 500 (FIG. 5) therethrough. The lower body portion 604 may further define, or otherwise provide, one or more tabs 606 (e.g., two tabs 606, however, only one visible in FIG. 6) receivable within a corresponding recess 506 (FIG. 5) of the drive interface 404 to removably couple the sterile barrier adapter 406 to the arm 402. The tabs 606 may be resiliently received in the recesses 506 (FIG. 5) to removably couple the sterile barrier adapter 406 to the drive interface 404. The tabs 606 can include grip surfaces 607 upon which a user can press to flex the tabs 606, thereby allowing the tabs 606 to be removed from the recesses 506 to allow the sterile barrier adapter 406 to be decoupled from the drive interface 404.

The upper body portion 602 may define one or more apertures 608 (e.g., two apertures 608) and the sterile barrier adapter 406 may further comprise one or more intermediate rotary drivers 610 that extend through (penetrate) a corresponding aperture 608. The bottom of each intermediate rotary driver 610 may provide or otherwise define a recess (not shown) sized to receive a corresponding rotary output driver 500 (FIG. 5). Each recess may define or provide teeth (not shown) configured to intermesh with the teeth 504 (FIG. 5) of the rotary output drivers 500 (FIG. 5), such that rotating the rotary output drivers 500 correspondingly rotates the intermediate rotary driver 610 when meshed therewith. The upper body portion 602 may further define a plurality of recesses 616 (e.g., two shown), described in more detail below.

Each intermediate rotary driver 610 includes a drive shaft 612 that defines, or otherwise provides, a plurality of teeth 614. While two intermediate rotary drivers 610 are shown and described, the sterile barrier adapter 406 could include less (e.g., one) or more (e.g., two, three, or four) than two intermediate rotary drivers 610. As illustrated, the number of intermediate rotary drivers 610 may be less than the number of rotary output drivers 500 (FIG. 5). However, other embodiments are envisioned where the number of intermediate rotary drivers 610 is the same as or greater than the number of rotary output drivers 500 (FIG. 5).

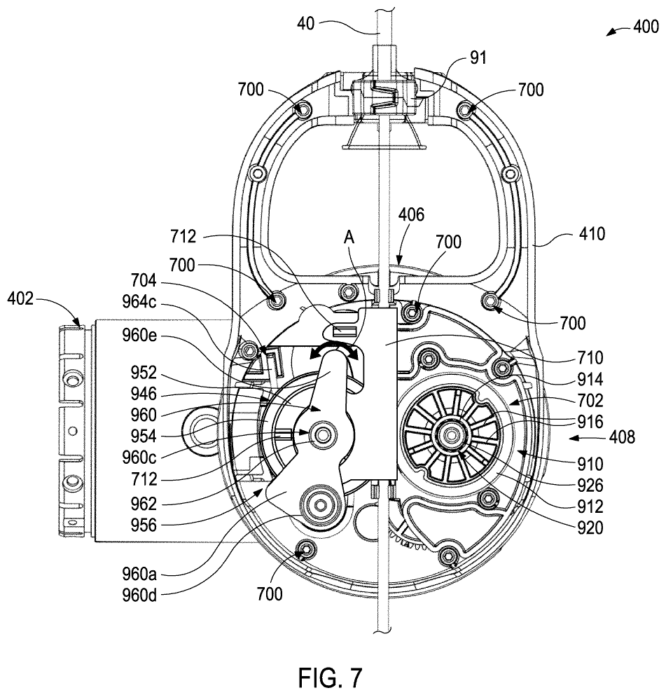

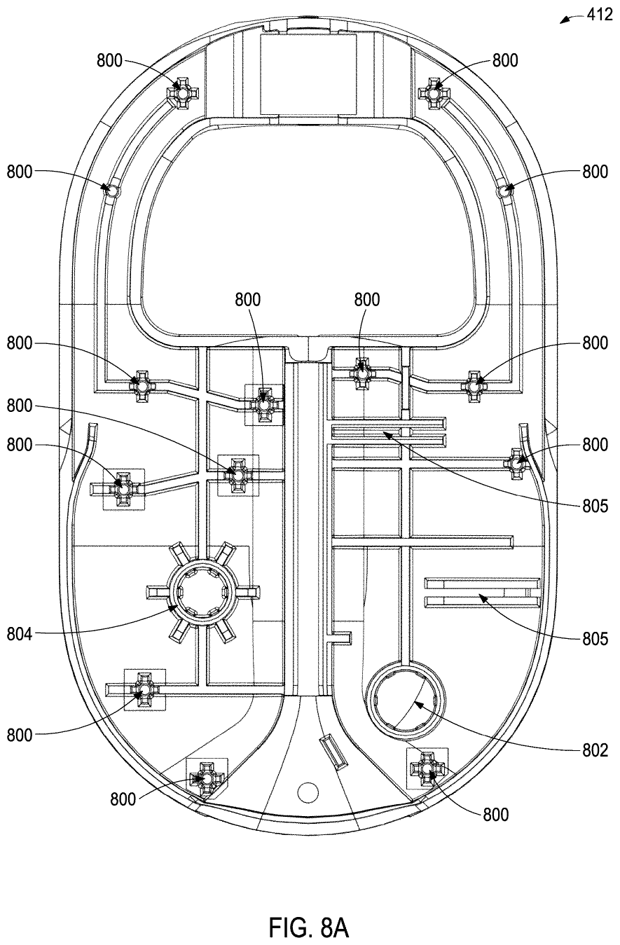

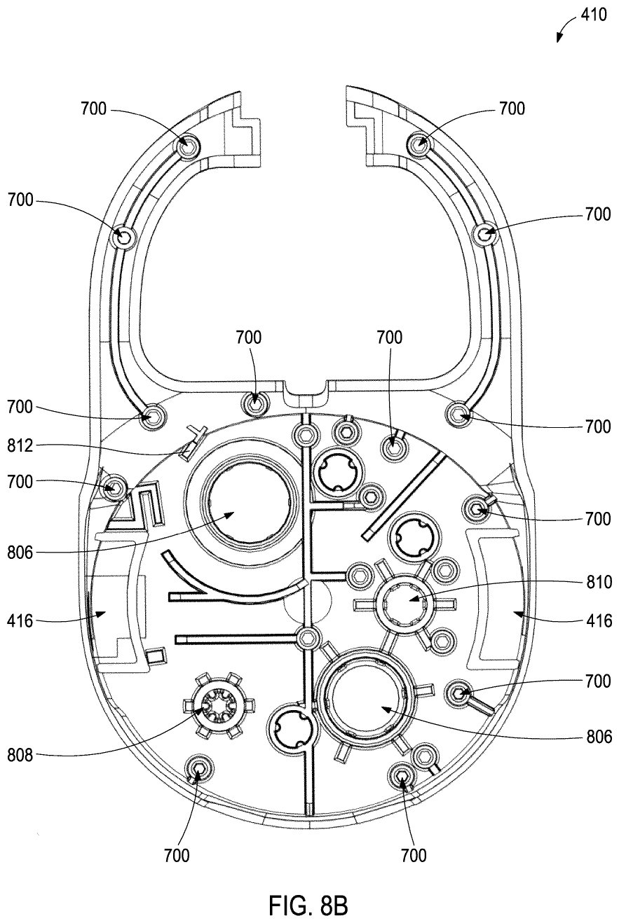

Referring now to FIGS. 4, 7, 8A, and 8B, the scope driver 400 includes a housing 408 that comprises a body 410 and a cover 412 coupled to the body 410. The cover 412 may be decoupled (removed) from the body 410 to expose an interior of the body 410 and the various components stored therein (see FIG. 7) or the cover 412 may be coupled to the body 410, such as with an adhesive, thereby preventing the cover 412 from being removed from the body 410. With particular reference to FIG. 8A, the cover 412 includes a plurality of pins 800, a first recess 802, a second recess 804, and a pair of grooves 805. With particular reference to FIGS. 7 and 8B, the body 410 defines or otherwise provides a plurality of slots 700, a plurality of apertures 806 (e.g., two shown), a post 808, a recess 810, and a blocker arm 812. The slots 700 are configured to receive the pins 800 of the cover 412 to couple the cover 412 to the body 410. The number of apertures 806 may correspond to the number of intermediate rotary drivers 610 (FIG. 6) and each aperture 806 may be sized to receive a corresponding intermediate rotary driver 610 (FIG. 6) therethrough. The body 410 and the cover 412 may cooperatively define a channel 411 (FIG. 4) to receive a scope 40 therethrough. Removal of the cover 412 from the body 410 may enable a user to gain access to the scope 40 in the event that the user desires to apply manual forces to the scope 40, such as a retraction or “bailout” force away from the patient 7 (FIG. 1) or an advancement force toward the patient 7 (FIG. 1). The scope driver 400 may further include a slide cover 710 (shown in phantom) that includes arms 712 sized to be received in the grooves 805 of the cover 412.

With reference again to FIG. 4, the body 410 may further define, or otherwise provide, a plurality of tabs 416 (e.g., two tabs 416, one visible in FIG. 4, both visible in FIG. 8B) that may be received in a corresponding recess 616 (FIG. 6) of the sterile barrier adapter 406 to removably couple the scope driver 400 to the sterile barrier adapter 406. The tabs 416 may be resiliently received in the recesses 616 (FIG. 6) to removably couple the scope driver 400 to the sterile barrier adapter 406. The tabs 416 can include or provide grip surfaces 418 upon which a user can press to flex the tabs 416, thereby allowing the tabs 416 to be removed from the recesses 616 (FIG. 6) to allow the scope driver 400 to be decoupled from the sterile barrier adapter 406.

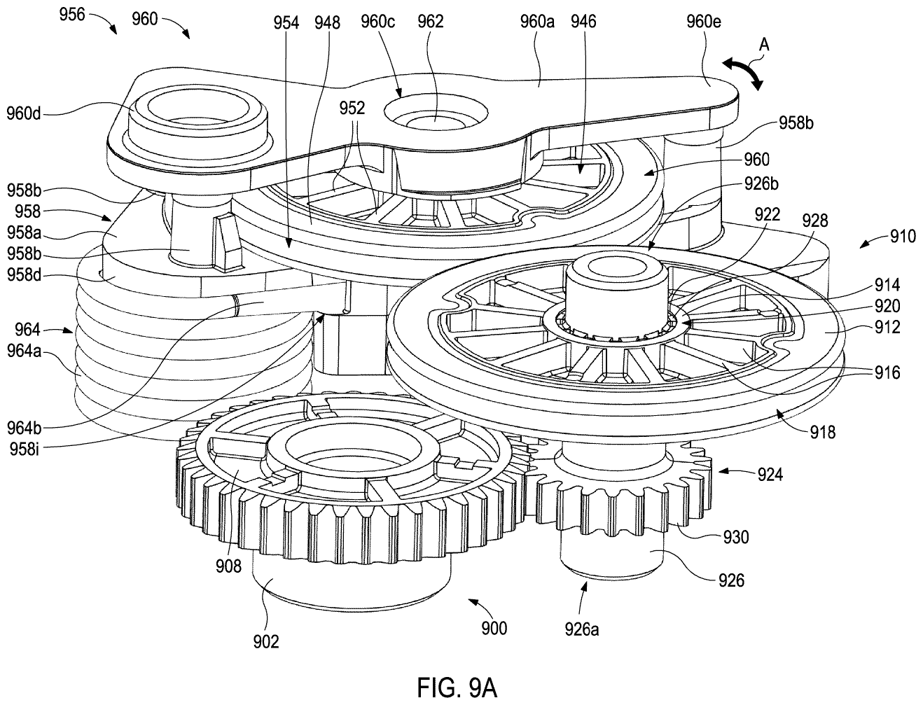

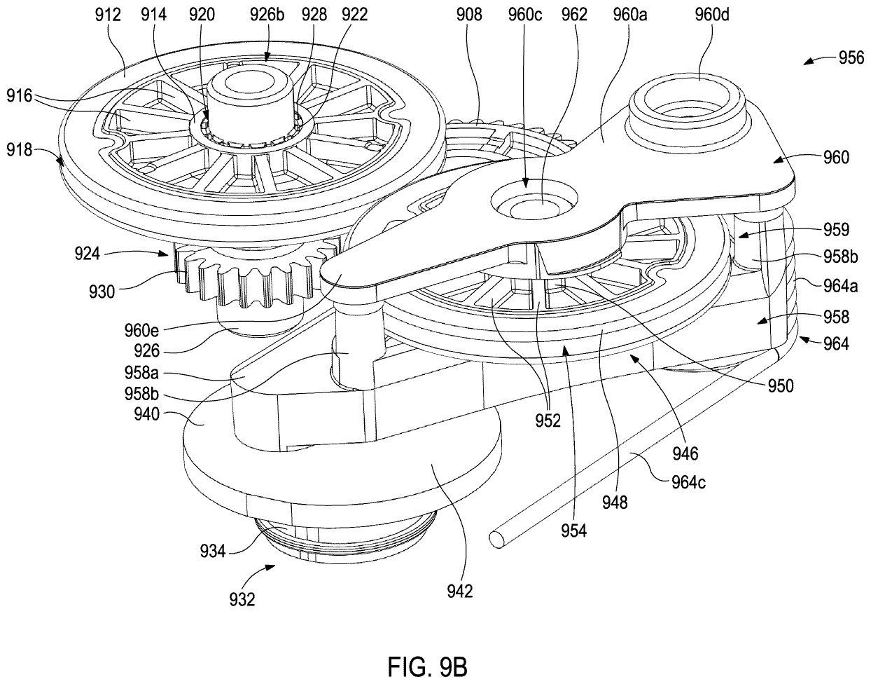

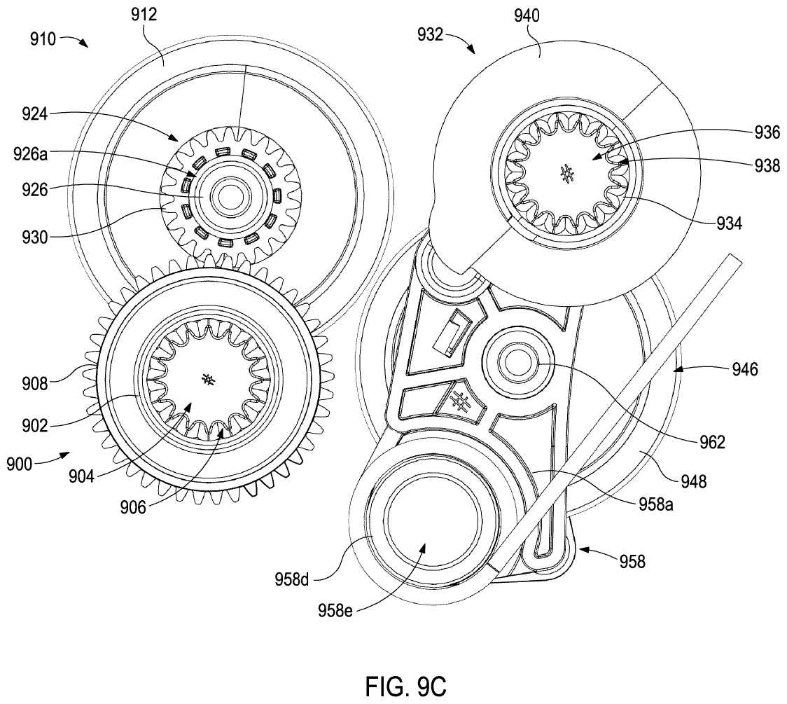

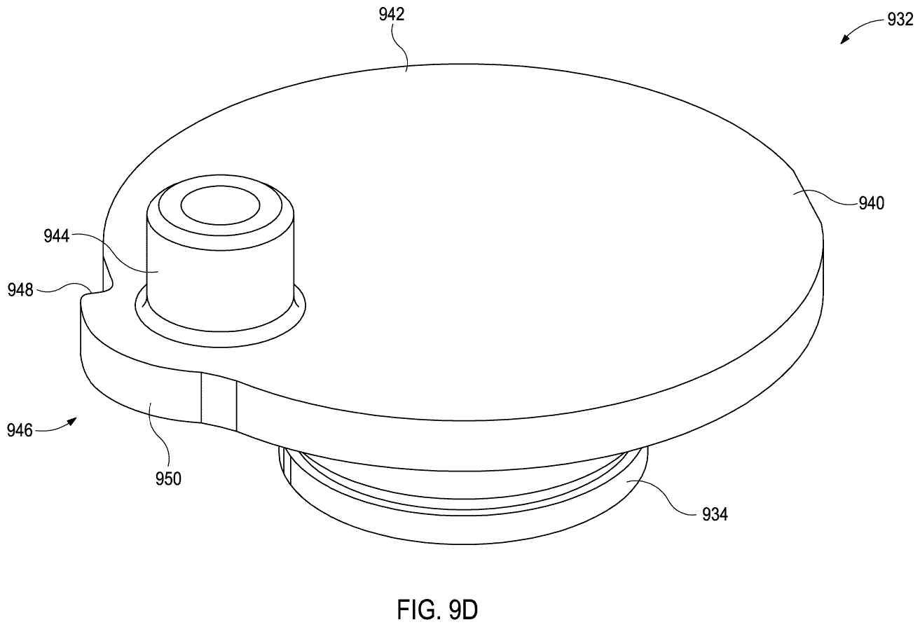

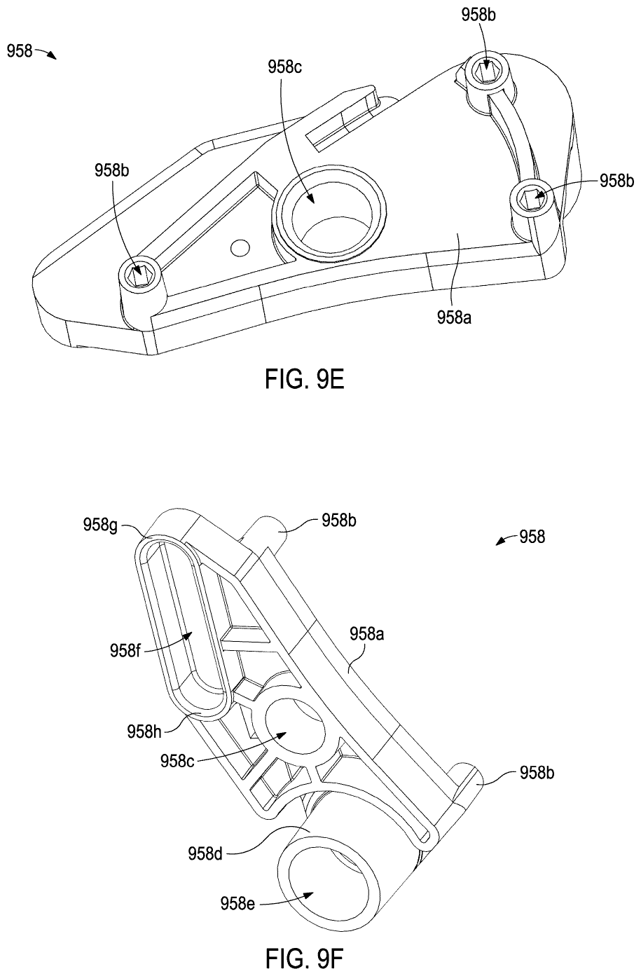

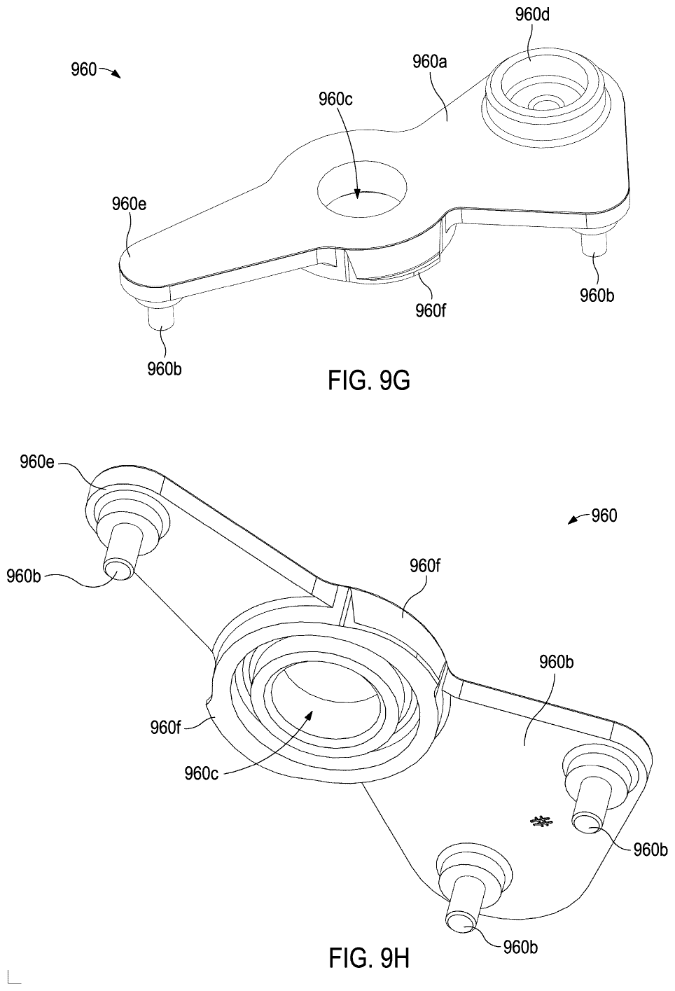

With reference now to FIGS. 7 and 9A-9H, the scope driver 400 includes a drive assembly 702 operable to drive the scope 40 within the channel 411 (FIG. 4) and relative to the scope driver 400. With particular reference to FIG. 9A, the drive assembly 702 includes a first rotary input driver 900 that comprises a shaft 902 defining a recess 904 (FIG. 9C) sized to receive a corresponding intermediate rotary driver 610 (FIG. 6) therein. The recess 904 provides teeth 906 that are configured to intermesh with the teeth 614 (FIG. 6) of the corresponding intermediate rotary driver 610, thereby allowing the intermediate rotary driver 610 to rotate the first rotary input driver 900 when meshed therewith.

The first rotary input driver 900 further includes a drive gear 908 extending from or otherwise mounted to the shaft 902. The shaft 902 may define a first diameter and the drive gear 908 may include teeth and define a second diameter different (e.g., greater) than the first diameter.

The drive assembly 702 further includes a drive wheel 910 that comprises an outer body portion 912, an inner body portion 914, and a plurality of spokes 916 extending between and coupling the inner and outer body portions 912, 914. The outer body portion 912 defines a groove or channel 918 sized to receive the scope 40 therein, as will be described in more detail below. The inner body portion 914 defines an aperture 920.

The drive assembly 702 further includes a pin 924 to operably couple the first rotary input driver 900 to the drive wheel 910 such that rotation of the first rotary input driver 900 correspondingly rotates the drive wheel 910. The inner body portion 914 may define, or otherwise provide, a plurality of spline teeth 922 that extend into the aperture 920. The pin 924 may include a body 926 that defines a plurality of spline teeth 928 to intermesh with the spline teeth 922, thereby allowing the pin 924 to rotate the drive wheel 910. Alternatively, the pin 924 may be positioned through the aperture 920 and may be coupled to the drive wheel 910, such as with an adhesive or with welding, such that rotation of the pin 924 causes corresponding rotation of the drive wheel 910. The body 926 includes a first end 926a receivable in the recess 810 (FIG. 8B) of the body 410, and a second end 926b receivable in the aperture 804 (FIG. 8A) of the cover 412 to rotatably couple the pin 924 to the housing 408.

The pin 924 further includes a driven gear 930 including teeth for intermeshing with the teeth of the drive gear 908, thereby allowing the first rotary input driver 900 to rotate the pin 924 and, consequently, the drive wheel 910 via the intermeshed spine teeth 922, 928. The gear 908 may allow the drive wheel 910 to rotate at a faster, slow, or the same speed as the intermediate rotary driver 610. As shown best in FIG. 7, the drive wheel 910 may be displaced from the scope 40 such that rotation of the drive wheel 910 alone does not cause the scope 40 to move relative to the scope driver 400, as discussed in more detail below.

With particular reference to FIG. 9B, the drive assembly 702 further includes a second rotary input driver 932 that comprises a shaft 934 defining a recess 936 (FIG. 9C) sized to receive a corresponding intermediate rotary driver 610 (FIG. 6) therein. The recess 936 provides teeth 938 that are configured to intermesh with the teeth 614 (FIG. 6) of the corresponding intermediate rotary driver 610, thereby allowing the intermediate rotary driver 610 to rotate the second rotary input driver 932 when meshed therewith.

The second rotary input driver 932 further includes a disk 940 extending from the shaft 934; e.g., radially outward. The shaft 934 may define a first diameter and the disk 940 may define a second diameter different (e.g., greater) than the first diameter. The disk 940 provides an upper surface 942, a pin 944 (see FIG. 9D) extending from the upper surface 942, and an arm 946 (see FIG. 9D). With reference to FIGS. 8B and 9D, the arm 946 defines a first of “blocker” surface 948 that may contact the blocker arm 812 of the body 410 when the second rotary input driver 932 is rotated in a first radial direction (e.g., clockwise, as viewing in FIG. 8B), thereby preventing the second rotary input driver 932 from rotating in the first radial direction. Conversely, the arm 946 further defines a second or “cam” surface 950 that may slide past the blocker arm 812 of the body when the second rotary input driver 932 is rotated in a second radial direction (e.g., counterclockwise, as viewing in FIG. 8B) opposite the first radial direction, thereby allowing the second rotary input driver 932 to rotate in the second radial direction.

The drive assembly 702 further includes an idler wheel 946, which may be substantially similar to the drive wheel 910. For instance, the idler wheel 946 includes an outer body portion 948, an inner body portion 950 (substantially occluded from view, but similar to the inner body portion 914), and a plurality of spokes 952 extending between and coupling the inner and outer body portions 948, 950. The outer body portion 948 defines a groove or channel 954 sized to receive the scope 40 therein, as will be described in more detail below. The inner body portion 966 defines an aperture (occluded from view, but similar to aperture 920).

The drive assembly 702 further includes a swing arm 956 that operably couples the second rotary input driver 932 to the idler wheel 946 such that movement (rotation) of the second rotary input driver 932 correspondingly moves (rotates) the idler wheel 946. More particularly, operation of the second rotary input driver 932 will cause the swing arm 956 to reciprocate, as indicated by the arrow A (FIGS. 7 and 9A), and thereby correspondingly move (pivot) the idler wheel 946 toward or away from drive wheel 910.

The swing arm 956 includes a first or “lower” swing arm portion 958 and a second or “upper” swing arm portion 960. The upper swing arm portion 960 includes a body 960a that defines, or otherwise provides, a plurality of posts 960b (FIG. 9H) extending from the underside of the body 960a, and the lower swing arm portion 958 includes a body 958a that defines, or otherwise provides, a plurality of recesses 958b (FIG. 9E) to receive a corresponding post 960b, thereby coupling the upper and lower swing arm portions 958, 960 together and defining a gap 959 (i.e., spacing) therebetween that is sized to receive the idler wheel 946.

The body 960a of the upper swing arm portion 960 further defines an aperture 960c, and the body 958a of the lower swing arm portion 958 further defines an aperture 958c (FIG. 9E) coaxially aligned with the aperture 960c when the upper and lower swing arm portions 958, 960 are mated. A pin 962 is extendable through the coaxially aligned apertures 958c, 960c, and the aperture of the idler wheel 946, thereby coupling the idler wheel 946 to the swing arm 956. The pin 962 enables the idler wheel 946 to rotate relative to the swing arm 956 while allowing the idler wheel 946 to be translated within the housing 408 by the swing arm 956, as will be explained in more detail below.

The swing arm 956 is rotatably mounted to the housing 408. For instance, the body 960a of the upper swing arm portion 960 defines or otherwise provides a post 960d to be received in the recess 802 (FIG. 8A) of the cover 412 (FIG. 8A), thereby rotatably coupling the upper swing arm portion 960 to the cover 412. The lower swing arm portion 958 defines, or otherwise provides, a post 958d (FIG. 9F) that defines a recess 958e (FIG. 9F) sized to receive the post 808 (FIG. 8B) of the body 410 (FIG. 8), thereby rotatably coupling the lower swing arm portion 958 to the body 410.

The body 960a of the upper swing arm portion 960 further defines, or otherwise provides, a pair of tabs 960f operable to engage a bottom surface of the slide cover 710 (FIG. 7) to suspend the slide cover 710 above the idler wheel 946.

With particular reference to FIGS. 9D and 9F, the lower swing arm portion 958 further defines, or otherwise provides, a slot 958f that includes a first end 958g and a second end 958h. The slot 958f is sized to receive the pin 944 of the second rotary input driver 932, and as the second rotary input driver 932 rotates, the pin 944 slides within the slot 958f between the first and second ends 958g, 958h. While sliding within the slot 958f, the pin 944 imparts (applies) forces to the lower swing arm portion 958 within the slot 958f, thereby causing the swing arm 956 to rotate about axially aligned posts 958d, 960d and reciprocate between a first or “disengaged” rotational position and a second or “engaged” rotational position. In the first rotational position, a first distance is defined between a tip 960e (FIG. 9G) of the upper swing arm portion 960 and the drive wheel 910, while in the second rotational position, a second distance less than the first distance is defined between the tip 960e of the upper swing arm portion 960 and the drive wheel 910.

Said differently, reciprocating the swing arm 956 causes the idler wheel 946 to move (reciprocate) toward or away from the drive wheel 910 and between first and second positions, as indicated by the arrow A (FIGS. 7 and 9A). In the first position, the idler wheel 946 is located at a first distance from the drive wheel 910, while in the second position, the idler wheel 946 is located at a second distance less than the first distance from the drive wheel 910. In the first position, the idler wheel 946 is displaced from (disengaged from) the scope 40, but moving the idler wheel 946 to the second position places the scope 40 in engagement between the drive and idler wheels 946. In some applications, in the first position, neither the drive wheel 910 nor the idler wheel 946 engage the scope 40. Accordingly, rotation of the drive wheel 910 with the idler wheel 946 in the first position does not yield movement of the scope 40.

Furthermore, as the swing arm 956 reciprocates, the swing arm applies lateral forces to the slide cover 710 via the tabs 960f. For instance, as the idler wheel 946 moves toward the first position, the swing arm 956 applies a lateral force in a first or “left” direction (as viewed in FIG. 7) to the slide cover 710. Conversely, as the idler wheel 946 moves toward the second position, the swing arm 956 applies a lateral force in a second or “right” direction (as viewed in FIG. 7) to the slide cover 710 via tabs 960f. Applying a lateral force to the slide cover 710 causes the slide cover 710 to move laterally with the motion of the swing arm 956, with the arms 712 of the slide cover 710 sliding in the grooves 805 (FIG. 8A) of the cover 412 (FIG. 8A), without inhibiting the rotational motion of the idler wheel 946.

As the idler wheel 946 is translated toward the second position, the idler wheel 946 receives the scope in the channel 954, and may drive the scope 40 into the opposing channel 918 of the drive wheel 910. Accordingly, in the second position, the scope 40 is engaged, or at least substantially engaged, with both (pinched between) the drive wheel 910 and the idler wheel 946. Once the scope 40 is pinched between the drive wheel 910 by the idler wheel 946, rotating the drive wheel 910 applies a frictional, translation force to the scope 40, which causes the scope 40 to axially translate. For instance, if the drive wheel 910 rotates in a first rotational direction (e.g., clockwise), the scope 40 is advanced in a first direction (e.g., a forward or distal direction). Moving the scope 40 in the first direction applies a frictional force to the idler wheel 946, which passively rotates about the pin 962 in a second rotational direction (e.g., counterclockwise) opposite the first rotational direction. Conversely, if the drive wheel 910 rotates in the second rotational direction, the scope 40 is advanced in a second direction opposite the first direction (e.g., a reverse or proximal direction). Moving the scope 40 in the second direction applies a frictional force to the idler wheel 946, which passively rotates about the pin 962 in the first rotational direction.

When translated toward and/or reaching the second position, the idler wheel 946 may, for a period of time, drive the scope 40 into engagement with the drive wheel 910 and apply a sufficient “pinching” force on the scope 40 to enable the drive wheel 910 to apply a frictional translation force to the scope 40. Conversely, when translated away from the second position and toward the first position, the idler wheel 946 may fail to provide the necessary force to the scope 40 to enable the drive wheel 910 to apply a frictional translation force to the scope 40. Accordingly, in some embodiments, the drive assembly 702 may progressively, or incrementally, displace the scope 40 through the channel 411 relative to the scope driver 400 as the idler wheel 946 is reciprocated (transitioned) between the first and second positions while the drive wheel 910 rotates. The periodic application of force to the scope 40 reduces the chance that the drive wheel 910 slips while translating the scope 40, which may increase the confidence of the control system 50 in identifying a position of the scope 40 within the patient 7.

The scope driver 400 further includes a spring 964 (FIGS. 9A and 9B). The spring 964 may function as a “toggle” to operably bias the swing arm 956 toward either the first rotational position, away from the drive wheel 910, or the second rotational position, toward the drive wheel 910. Accordingly, the spring 964 reliably positions the swing arm 956 in the first or second rotational position as opposed to “floating” in a rotational position therebetween. The spring 964 may comprise a torsion spring that includes a coiled portion 964a, a first end 964b, and a second end 964c. The coiled portion 964a is wound around the post 958d of the lower swing arm portion 958. The first end 964b is received in a slot 958i (FIG. 9A) defined in the lower swing arm portion 958 and the second arm 964c is received in a slot 704 (FIG. 7) defined in the body 410. The spring 964 may provide an amount of passive pinch force that increases the force that the idler wheel 946 applies to the scope 40, thereby preventing, or at least substantially reducing, the chance of slip between the idler wheel 946 and the scope 40. This arrangement helps reduce heat buildup and wear in the motor and eliminates the need for a computational torque feedback system to achieve constant pinch.

In some embodiments, the drive assembly 702 may include a multi-bar linkage or cam mechanism for applying a pinching force on the scope 40. In some embodiments, the pinching force may be achieved with a motor where the motor torque is scaled up by a gearing mechanism or other mechanical advantage. In some embodiments, the drive assembly 702 may further include a tread or a belt mechanism, such as on the drive and idler wheels 910, 946, which may yield increased grip forces on the scope 40 when engaged therewith. In some embodiments, the scope driver 400 may include manual override system that includes a break-away mechanism for permanently disengaging the drive assembly 702 from the corresponding motors in the arm 402.

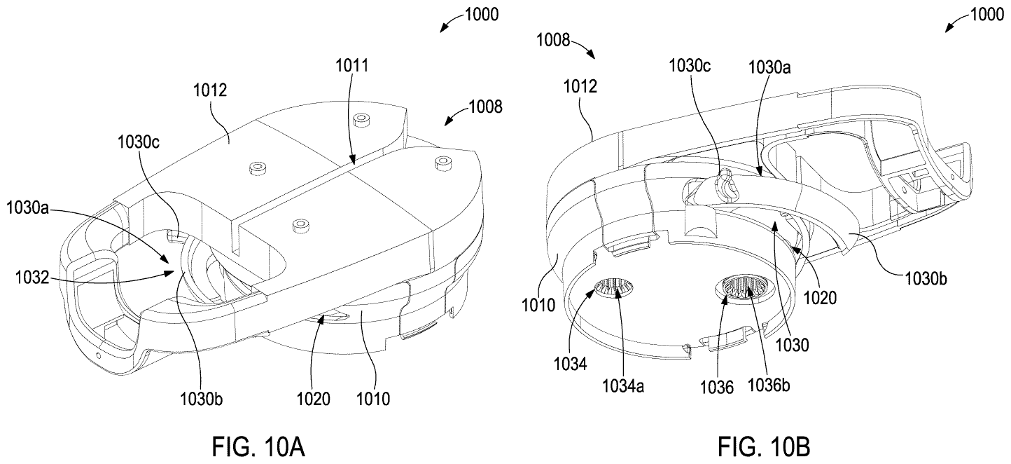

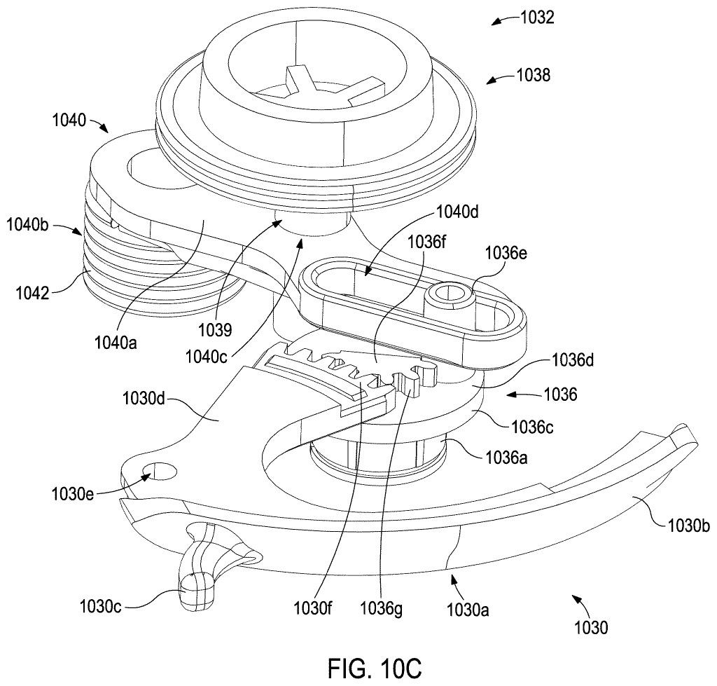

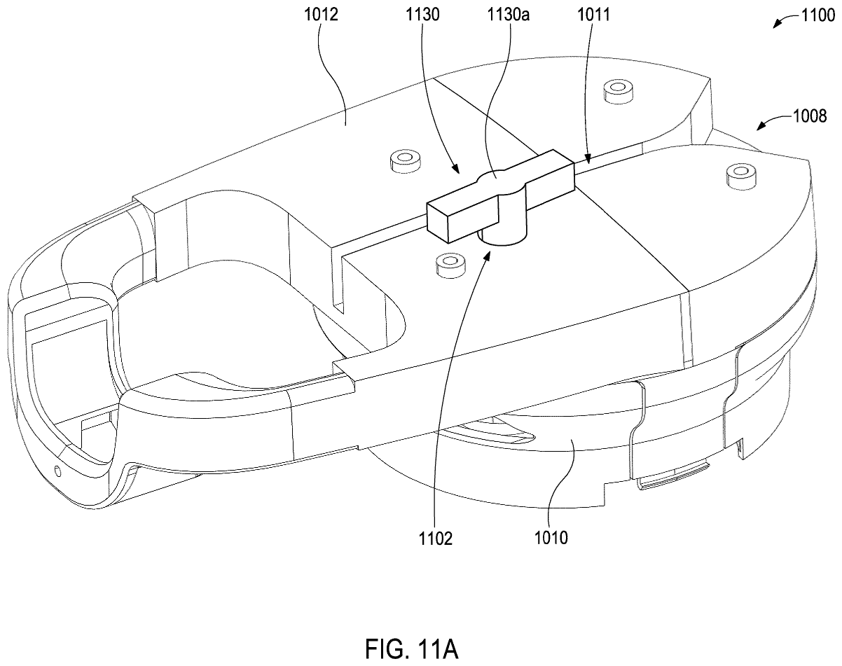

FIGS. 10A and 10B are isometric views of an example scope driver 1000, in accordance with at least one aspect of the present disclosure. FIG. 10A is an isometric view of the scope driver 1000 from a top right perspective, and FIG. 10B is an isometric view of the scope driver 1000 from a bottom left perspective. The scope driver 1000 may be similar in some respects to the scope driver 400 of FIG. 4, and therefore may be best understood with reference thereto.

The scope driver 1000 includes a housing 1008, similar to housing 408 (FIG. 4), that comprises a body 1010, similar to body 410 (FIG. 4), and a cover 1012, similar cover 412 (FIG. 4), coupled to the body 1010. The cover 1012 may be removable from the body 1010 to expose an interior of the body 1010 and the various components stored therein. Alternatively, the cover 1012 may be coupled to the body 1010 such as with an adhesive or welding, thereby preventing the cover 1012 from being removed from the body 1010. The body 1010 and the cover 1012 may cooperatively define a channel 1011 to receive a scope, like scope 40 (FIG. 1), therethrough.

The body 1010 of the housing 1008 defines an aperture 1020 that extends around a portion of the circumference of the body 1010. The scope driver 1000 may further include a bailout 1030 that includes a lever 1030a that extends at least partially into the aperture 1020 and that is rotatably (pivotably) coupled to the body 1010, thereby allowing the lever 1030 to be rotated relative to the housing 1008. The lever 1030a may include a grip 1030b that is graspable (engageable) by a user to rotate the lever 1030a. The lever 1030a may provide an extension 1030c that extends from the grip 1030b and that provides a location for the user to position their thumb when rotating the lever 1030a, which may aid in providing additional torque (mechanical advantage) when actuating (rotating) the lever 1030a. For instance, the user may wrap their index, middle, ring, and pinky fingers around the grip 1030b and press their thumb against the extension 1030c.

FIG. 10C is an isometric view of the bailout 1030 and a portion of a drive assembly 1032 of the scope driver 1000, in accordance with at least one aspect of the present disclosure. The drive assembly 1032 may be similar to the drive assembly 702 (FIG. 7). For instance, with reference to FIGS. 10B and 10C, the drive assembly 1032 includes a first rotary input driver 1034, which may be similar to the first rotary input driver 900 (FIG. 9A), that is rotatably mounted to the body 1010 and that includes a shaft (not shown, but similar to shaft 902 of FIG. 9A) defining a recess 1034a sized to receive an intermediate rotary driver 610 (FIG. 6) therein. The intermediate rotary driver 610 may rotate the first rotary input driver 1034 when meshed therewith to rotate a drive wheel (not shown, but similar to drive 910 of FIG. 9A) of the drive assembly 1032.

With continued reference to FIGS. 10B and 10C, the drive assembly 1032 further includes a second rotary input driver 1036, which may be similar to the second rotary input driver 932 (FIG. 9B), that is rotatably mounted to the body 1010 and that includes a shaft 1036a that defines a recess 1036b sized to receive an intermediate rotary driver 610 (FIG. 6) therein. The second rotary input driver 1036 further includes a disk 1036c that extends radially outward from the shaft 1036a. The shaft 1036a may define a first diameter and the disk 1036c may define a second diameter different (e.g., greater) than the first diameter. The disk 1036c provides an upper surface 1036d and a pin 1036e extends from the upper surface 1036d. A gear 1036f is mounted to or otherwise forms part of the upper surface 1036d and provides first teeth 1036g. The second rotary input driver 1036 may further include an arm (not shown, but similar to arm 946 of FIG. 9D) for controlling the rotational direction that the second rotary input driver 1036 rotates within the housing 1008.

The drive assembly 1032 further includes an idler wheel 1038, which may be similar to the idler wheel 946 (FIG. 9A). In combination with the drive wheel of the drive assembly 1032, the idler wheel 1038 functions to drive (translate) a scope (e.g., scope 40; FIG. 1) proximally or distally within the channel 1011, relative to the scope driver 1000.

The drive assembly 1032 further includes a swing arm 1040, which may be similar to the swing arm 956 (FIG. 9A), that operatively couples the second rotary input driver 1036 to the idler wheel 1038 such that rotation of the second rotary input driver 1036 reciprocates (moves) the idler wheel 1038 toward and away from the drive wheel. More specifically, the swing arm 1040 includes a body 1040a and a post 1040b (substantially occluded from view, but similar to post 958d; FIG. 9F) extending from the body 1040a. The post 1040b defines a recess, like recess 958e (FIG. 9F), for receiving a post, like post 808 (FIG. 8B) of the body 1010, thereby rotatably coupling the swing arm 1040 to the body 1010. The body 1040a defines an aperture 1040c through which a shaft 1039 extends, and the idler wheel 1038 is rotatably mounted to the shaft 1039, thereby allowing the swing arm 1040 to translate (move) the idler wheel 1038 and allowing the idler wheel 1038 to rotate relative to the shaft 1039. The idler wheel 1038 may provide a pin (not shown) and the shaft 1039 may define a recess (not shown) to receive the pin to rotatably couple the idler wheel 1038 to the shaft 1039.

The body 1040a of the swing arm 1040 defines a slot 1040d sized to receive the pin 1036e of the second rotary input driver 1036. As the second rotary input driver 1036 rotates, the pin 1036e slides within (traverses) the slot 1040d. While sliding within the slot 1040d, the pin 1036e imparts (applies) forces to the swing arm 1040 within the slot 1040d, thereby causing the swing arm 1040 to rotate about the post 1040b and reciprocate between a first or “disengaged” rotational position, in which the idler wheel 1038 is displaced from the scope and the drive wheel, and a second or “engaged” rotational position, in which the idler wheel 1038 and drive wheel “pinch” (engage) the scope to translate the scope relative to the scope driver 1000.

The scope driver 1000 may further include a spring 1042, which be similar to spring 964 (FIG. 9A), that is positioned around the post 1040b and which may function as a “toggle” to bias the swing arm 1040 toward or away from the drive wheel, thereby reliably positioning the swing arm 1040 in two potential rotational positions as opposed to “floating” in a rotational position therebetween.

The lever 1030a further includes a body 1030d that extends from the grip 1030b. The body 1030d defines an aperture 1030e to receive a pin (not shown) of the housing 1008, which rotatably couples the lever 1030a to the housing 1008. The body 1030d may provide second teeth 1030f arranged to mesh with the first teeth 1036g of the gear 1036f.

During use of the scope drive 1000, a user may desire to remove the scope vertically (upwardly) from the channel 1011. However, the spring 1042 may naturally bias the swing arm 1040 to the second or “engaged” rotational position, thereby pinching the scope between the drive wheel and idler wheel 1038, preventing the user from removing the scope from the channel 1011. To remove the scope from the channel 1011, the user may grasp the lever 1030a and rotate the grip 1030b away from the aperture 1020. Rotating the grip 1030b drives the second teeth 1030f against the first teeth 1036g, thereby causing the second rotary input driver 1036 to rotate (e.g., counterclockwise, as viewed in FIG. 10C). Rotating the second rotary input driver 1036 causes the swing arm 1040 to rotate about the post 1040b toward the first or “disengaged” rotational position, in which the idler wheel 1038 is displaced from the scope and the drive wheel. With the swing arm 1040 in the disengaged rotational position, the user may remove the scope vertically (upwardly) from the channel 1011.

In some embodiments, the first and second teeth 1036g, 1030f are meshed at all times after the scope driver 1000 has been assembled. In other embodiments, the bailout 1030 may be transitionable between a first state, in which the first and second teeth 1036g, 1030f are meshed, as shown in FIG. 10C, and a second state, in which the first and second teeth 1036g, 1030f are demeshed (disengaged). For instance, with reference to FIGS. 10B and 10C, to transition the bailout 1030 to the second state, the lever 1030a may be rotated toward the body 1010 until the grip 1030b occludes, or substantially occludes, the aperture 1020, thereby causing the second teeth 1030f to rotate beyond, and demesh (disengage) from, the first teeth 1036g. To transition back to the first state, a user may rotate the lever 1030a away from the body 1010, causing the grip 1030b to move out of the aperture 1020, and the first and second teeth 1036g, 1030f to remesh (re-engage) with one another. Alternatively, to transition the bailout 1030 to the second state, the lever 1030a may be translated away from the gear 1036f to demesh the first and second teeth 1036g, 1030f.

Accordingly, the bailout 1030 enables a user to remove the scope from the scope driver 1000 in the vertical (upward) direction without needing to disassemble the scope driver 1000 (e.g., removing the cover 1012 from the body 1010 and navigating the various internals of the scope driver 1000 to manually move the swing arm 1040 away from the scope).

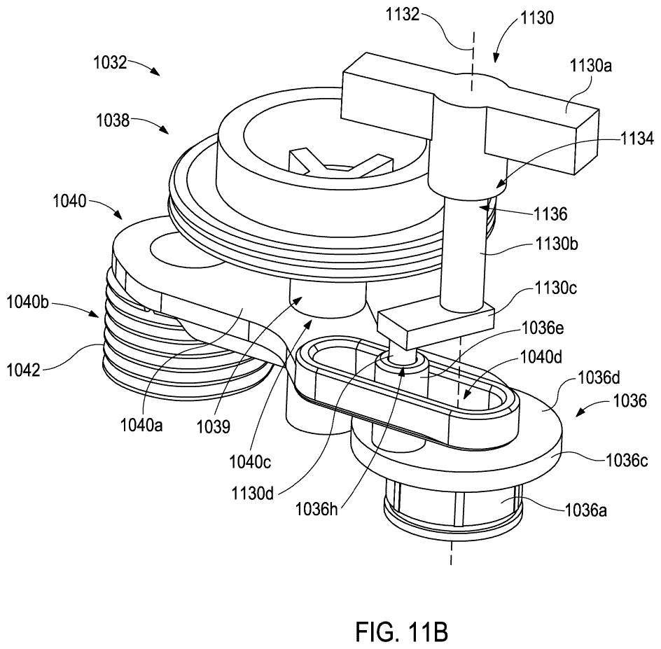

FIG. 11A is an isometric view of another example scope driver 1100 from a top right perspective, in accordance with at least one aspect of the present disclosure. The scope driver 1100 may be similar in some respects to the scope driver 400 (FIG. 4) and the scope drive 1000 (FIG. 11A), and therefore may be best understood with reference thereto, where like numerals will correspond to like components not described again in detail.

The cover 1012 of the housing 1008 may define an aperture 1102 that extends into the interior of the housing 1008. The scope driver 1100 may further include a bailout 1130 that includes a wingnut 1130a that extends through the aperture 1020 and is manually rotatable by a user, as will be explained in more detail below.

FIG. 11B is an isometric view of the bailout 1130 and a portion of the drive assembly 1032 of the scope driver 1100, in accordance with at least one aspect of the present disclosure. As illustrated, the bailout 1130 includes a drive shaft 1130b that extends from the wingnut 1130a in a first direction (e.g., downward), a base 1130c that extends from the drive shaft 1130b in a second direction (e.g., laterally) different than (e.g., orthogonal to) the first direction, and a pin 1130d that extends from the base 1130c in the first direction. The drive shaft 1130b, the base 1130c, and the pin 1130d may be of unitary construction, or may be separate components coupled together, such as with welding or a mechanical attachment, thereby non-rotatably coupling the drive shaft 1130b, the base 1130c, and the pin 1130d together. The pin 1036e of the second rotary input driver 1036 defines a recess 1036h that is sized to receive the pin 1130d of the bailout 1130 therein. The drive shaft 1130a defines an axis 1132 that extends through a center point of (is co-axial with) the second rotary input drive 1036. Accordingly, the drive shaft 1130a and the second rotary input driver 1036 both rotate about the axis 1132 when the bailout 1130 is actuated, as will be described in more detail below.

The wingnut 1130a may be removably coupled to (disengageable from) the drive shaft 1130b, thereby allowing the bailout 1130 to be transitioned between a first state, in which rotation of the wingnut 1130a causes rotation of the drive shaft 1130b, and a second state, in which rotation of the wingnut 1130a does not cause rotation of the drive shaft 1130b. For instance, a lower end 1134 of the wingnut 1130a may define a recess (not shown) that includes first splines (not shown) and an upper end 1136 of the drive shaft 1130b may define second splines (not shown) that are meshable with the first splines such that rotation of the wingnut 1130a causes corresponding rotation of the drive shaft 1130b. In the first state, the upper end 1136 of the drive shaft 1130b may be received in the recess at the lower end 1134 of the wingnut 1130a to mesh the first and second splines together. In the second state, the wingnut 1130a may be lifted (manually by a user) off the upper end 1136 of the drive shaft 1130b, desmeshing the first and second splines and exposing the upper end 1136 of the drive shaft 1130b. Removing the wingnut 1130a may prevent a user from inadvertently actuating the bailout 1130 while the scope driver 1100 is in use. Alternatively, the wingnut 1130a is fixedly coupled to the drive shaft 1130b, such as with welding or an adhesive, such that the bailout 1130 cannot be transitioned to the second state.

During use of the scope drive 1100, a user may desire to remove the scope vertically (upwardly) from the channel 1011. However, due to the spring 1042 biasing the swing arm 1040 to the second or “engaged” rotational position, the drive wheel and the idler wheel 1038 may co-operatively pinch the scope, thereby preventing the user from removing the scope from the channel 1011. To remove the scope, the user may grasp and rotate the wingnut 1130a, causing the drive shaft 1130b, the base 1130c, and the pin 1130d to rotate about the axis 1132. Rotation of the pin 1130d causes the pin 1130d to apply a force to the pin 1036e of the second rotary input driver 1036, thereby causing the second rotary input driver 1036 to similarly rotate about the axis 1132. Rotating the second rotary input driver 1036 about the axis 1132 causes the swing arm 1040 to rotate about the post 1040b toward the first or “disengaged” rotational position, in which the idler wheel 1038 is displaced from the scope and the drive wheel. With the swing arm 1040 in the disengaged rotational position, the user may remove the scope vertically (upwardly) from the channel 1011.

Accordingly, the bailout 1130 enables a user to remove the scope from the scope driver 1100 in the vertical (upward) direction without needing to disassemble the scope driver 1100 (e.g., removing the cover 1012 from the body 1010 and navigating the various internals of the scope driver 1100 to manually move the swing arm 1040 away from the scope).

Embodiments disclosed herein include:
A. A scope driver comprising a housing defining a channel sized to receive a scope, a first rotary input driver rotatably mounted to the housing and drivable by a first rotary output driver of a robotic system, a drive wheel arranged within the housing and operatively coupled to the first rotary input driver such that rotation of the first rotary input driver correspondingly rotates the drive wheel, a second rotary input driver rotatably mounted to the housing and drivable by a second rotary output driver of the robotic system, and an idler wheel mounted to a swing arm within the housing and operatively coupled to the second rotary input driver such that actuation of the second rotary input driver correspondingly reciprocates the idler wheel toward or away from the drive wheel, wherein moving the idler wheel toward the drive wheel engages the scope between the drive and idler wheels, and wherein moving the idler wheel away from the drive wheel disengages the scope from at least one of the drive and idler wheels.
B. A scope driver comprising a housing defining a channel sized to receive a scope, a drive wheel rotatably mounted within the housing, a swing arm rotatably mounted within the housing, and an idler wheel mounted to the swing arm within the housing, the swing arm being operable to reciprocate the idler wheel between a first position in which the idler wheel is displaced from the scope and a second position in which the idler wheel drives the scope into engagement with the drive wheel, thereby allowing the drive wheel to engage and axially translate the scope within the channel.
C. A method comprising arranging a scope within a channel defined by a housing of a scope driver, the scope driver further including a first rotary input driver rotatably mounted to the housing, a drive wheel arranged within the housing and operatively coupled to the first rotary input driver such that rotation of the first rotary input driver correspondingly rotates the drive wheel, a second rotary input driver rotatably mounted to the housing, and an idler wheel mounted to a swing arm within the housing and operatively coupled to the second rotary input driver such that actuation of the second rotary input driver correspondingly reciprocates the idler wheel toward or away from the drive wheel, reciprocating the swing arm of the scope driver from a first rotational position toward a second rotational position and thereby moving the idler wheel toward the drive wheel to engage the scope between the drive and idler wheels, and rotating the drive wheel and thereby incrementally translating the scope through the channel.
D. A scope driver comprising a housing defining a channel sized to receive a scope, a rotary input driver rotatably mounted to the housing and drivable by a rotary output driver of a robotic system, a swing arm rotatably mounted to the housing and rotatable based on rotation of the rotary input driver, an idler wheel mounted to the swing arm, the idler wheel being movable relative to the scope based on rotation of the swing arm, and a bailout operatively coupled to the idler wheel and actuatable to move the idler wheel away from the scope.

Each of embodiments A-D may have one or more of the following additional elements in any combination: Element 1: wherein the swing arm includes a first swing arm portion and a second swing arm portion operatively coupled to the first swing arm portion to define a gap therebetween, wherein the idler wheel is rotatably mounted to the swing arm within the gap. Element 2: wherein the first swing arm portion defines a channel, and the second rotary input driver includes a pin translatable within the channel as the second rotary output driver actuates to move the idler wheel. Element 3: wherein a first aperture is defined by the first swing arm portion, a second aperture is defined by the second swing arm portion, and a third aperture is defined by the idler wheel, wherein the first, second, and third apertures are coaxially aligned within the housing, and wherein the scope driver further comprises a pin extending through the first, second, and third apertures to thereby allow the idler wheel to rotate relative to the swing arm. Element 4: further comprising a pin provided by the second swing arm portion and an aperture defined by the housing and sized to receive the pin to thereby rotatably couple the swing arm to the housing. Element 5: further comprising a spring to bias the swing arm and thereby urge the idler wheel toward and away from the drive wheel. Element 6: wherein the spring comprises a first end receivable within a first slot defined by the housing and a second end receivable within a second slot defined by the swing arm. Element 7: wherein the idler wheel is translatable by the swing arm between a first position in which the idler wheel is displaced from the scope and a second position in which the idler wheel engages the scope. Element 8: wherein rotation of the drive wheel causes passive rotation of the idler wheel based on the drive and idler wheels engaging the scope. Element 9: wherein the drive and idler wheels each define channels sized to receive and engage the scope when the idler wheel is moved to the second position. Element 10: wherein, in the second position, rotation of the drive wheel causes passive rotation of the idler wheel. Element 11: wherein the swing arm includes a first swing arm portion and a second swing arm portion operatively coupled to the first swing arm portion to define a gap therebetween, wherein the idler wheel is rotatably mounted to the swing arm within the gap. Element 12: wherein the first swing arm portion defines a channel, and the scope driver further comprises a rotary input driver that includes a pin translatable within the channel as the second rotary output driver actuates to move the idler wheel. Element 13: wherein a first aperture is defined by the first swing arm portion, a second aperture is defined by the second swing arm portion, and a third aperture is defined by the idler wheel, wherein the first, second, and third apertures are coaxially aligned within the housing, and wherein the scope driver further comprises a pin extending through the first, second, and third apertures to thereby allow the idler wheel to rotate relative to the swing arm. Element 14: further comprising a pin provided by the second swing arm portion and an aperture defined by the housing and sized to receive the pin to thereby rotatably couple the swing arm to the housing. Element 15: further comprising a spring to bias the swing arm and thereby urge the idler wheel toward and away from the drive wheel. Element 16: wherein the spring comprises a first end receivable within a first slot defined by the housing and a second end receivable within a second slot defined by the swing arm. Element 17: further comprising passively rotating the idler wheel as the drive wheel rotates. Element 18: wherein the bailout comprises a lever rotatably coupled to the housing. Element 19: wherein the housing defines an aperture and the lever is at least partially extendable through the aperture. Element 20: wherein the housing comprises a body and the aperture extends around a portion of a circumference of the body. Element 21: wherein the lever provides first teeth, and the rotary input driver comprises a gear that provides second teeth meshable with the first teeth. Element 22: wherein the lever is transitionable between a first state, in which the first teeth are meshed with the second teeth such that rotation of the lever causes rotation of the rotary input driver and a second state, in which the first teeth are demeshed from the second teeth such that rotation of the lever does not cause rotation of the rotary input driver. Element 23: wherein the bailout comprises a wingnut. Element 24: wherein the housing defines an aperture and a portion of the wingnut extends through the aperture. Element 25: wherein the bailout further comprises a drive shaft extending from the wingnut. Element 26: wherein the bailout is transitionable from a first state, in which the wingnut is coupled to the drive shaft and a second state, in which the wingnut is decoupled from the drive shaft. Element 27: wherein the drive shaft defines an axis that extends through a center point of the rotary input driver. Element 28: wherein the drive shaft extends in a first direction and the bailout further comprises a base extending in a second direction different than the first direction and a pin extending in the first direction. Element 29: wherein the swing arm defines a slot, and the rotary input driver provides a pin positioned and translatable within the slot. Element 30: wherein the pin of the rotary input driver defines a recess to receive the pin of the bailout. Element 31: wherein the rotary input driver is a first rotary input driver and the scope driver further comprises a second rotary input driver rotatably mounted to the housing and drivable by a second rotary output driver of the robotic system and a drive wheel arranged within the housing and operatively coupled to the first rotary input driver such that rotation of the first rotary input driver correspondingly rotates the drive wheel.

By way of non-limiting example, exemplary combinations applicable to A, B, C, and D include: Element 1 with Element 2; Element 1 with Element 3; Element 1 with Element 4; Element 5 with Element 6; Element 7 with Element 8; Element 7 with Elements 8 and 9; two or more of Elements 1-9; Element 11 with Element 12; Element 11 with Element 13; Element 11 with Element 14; Element 15 with Element 16; two or more of Elements 10-16; Element 18 with Element 19; Element 18 with Elements 19 and 20; Element 18 with Element 21; Element 18 with Elements 21 and 22; Element 23 with Element 24; Element 23 with Element 25; Element 23 with Elements 25 and 26; Element 23 with Elements 25 and 27; Element 23 with Elements 25 and 28; Element 23 with Elements 25, 28, and 29; Element 23 with Elements 25, 28, 29, and 30; two of more of Elements 18-31.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A scope driver, comprising:
a housing defining a channel sized to receive a scope;
a first rotary input driver rotatably mounted to the housing and drivable by a first rotary output driver of a robotic system;
a drive wheel arranged within the housing and operatively coupled to the first rotary input driver such that rotation of the first rotary input driver correspondingly rotates the drive wheel;
a second rotary input driver rotatably mounted to the housing and drivable by a second rotary output driver of the robotic system; and
an idler wheel mounted to a swing arm within the housing and operatively coupled to the second rotary input driver such that actuation of the second rotary input driver correspondingly reciprocates the idler wheel toward or away from the drive wheel,
wherein moving the idler wheel toward the drive wheel engages the scope between the drive and idler wheels, and
wherein moving the idler wheel away from the drive wheel disengages the scope from at least one of the drive and idler wheels.

2. The scope driver of claim 1, wherein the swing arm includes:
a first swing arm portion; and
a second swing arm portion operatively coupled to the first swing arm portion to define a gap therebetween,
wherein the idler wheel is rotatably mounted to the swing arm within the gap.

3. The scope driver of claim 2, wherein the first swing arm portion defines a channel, and the second rotary input driver includes a pin translatable within the channel as the second rotary output driver actuates to move the idler wheel.

4. The scope driver of claim 2, wherein:
a first aperture is defined by the first swing arm portion;
a second aperture is defined by the second swing arm portion; and
a third aperture is defined by the idler wheel, wherein the first, second, and third apertures are coaxially aligned within the housing; and
wherein the scope driver further comprises a pin extending through the first, second, and third apertures to thereby allow the idler wheel to rotate relative to the swing arm.

5. The scope driver of claim 2, further comprising:
a pin provided by the second swing arm portion; and
an aperture defined by the housing and sized to receive the pin to thereby rotatably couple the swing arm to the housing.

6. The scope driver of claim 1, further comprising a spring to bias the swing arm and thereby urge the idler wheel toward and away from the drive wheel.

7. The scope driver of claim 6, wherein the spring comprises:
a first end receivable within a first slot defined by the housing; and
a second end receivable within a second slot defined by the swing arm.

8. The scope driver of claim 1, wherein the idler wheel is translatable by the swing arm between:
a first position in which the idler wheel is displaced from the scope; and
a second position in which the idler wheel engages the scope.

9. The scope driver of claim 8, wherein rotation of the drive wheel causes passive rotation of the idler wheel based on the drive and idler wheels engaging the scope.

10. The scope driver of claim 9, wherein the drive and idler wheels each define channels sized to receive and engage the scope when the idler wheel is moved to the second position.

11. A scope driver, comprising:
a housing defining a channel sized to receive a scope;
a drive wheel rotatably mounted within the housing;
a swing arm rotatably mounted within the housing; and
an idler wheel mounted to the swing arm within the housing, the swing arm being operable to reciprocate the idler wheel between:
a first position in which the idler wheel is displaced from the scope; and
a second position in which the idler wheel drives the scope into engagement with the drive wheel, thereby allowing the drive wheel to engage and axially translate the scope within the channel.

12. The scope driver of claim 11, wherein, in the second position, rotation of the drive wheel causes passive rotation of the idler wheel.

13. The scope driver of claim 11, wherein the swing arm includes:
a first swing arm portion; and
a second swing arm portion operatively coupled to the first swing arm portion to define a gap therebetween,
wherein the idler wheel is rotatably mounted to the swing arm within the gap.

14. The scope driver of claim 13, wherein the first swing arm portion defines a channel, and the scope driver further comprises a rotary input driver that includes a pin translatable within the channel as the second rotary output driver actuates to move the idler wheel.

15. The scope driver of claim 13, wherein:
a first aperture is defined by the first swing arm portion;
a second aperture is defined by the second swing arm portion; and
a third aperture is defined by the idler wheel, wherein the first, second, and third apertures are coaxially aligned within the housing; and
wherein the scope driver further comprises a pin extending through the first, second, and third apertures to thereby allow the idler wheel to rotate relative to the swing arm.

16. The scope driver of claim 13, further comprising:
a pin provided by the second swing arm portion; and
an aperture defined by the housing and sized to receive the pin to thereby rotatably couple the swing arm to the housing.

17. The scope driver of claim 11, further comprising a spring to bias the swing arm and thereby urge the idler wheel toward and away from the drive wheel.

18. The scope driver of claim 17, wherein the spring comprises:
a first end receivable within a first slot defined by the housing; and
a second end receivable within a second slot defined by the swing arm.

19. A method, comprising:
arranging a scope within a channel defined by a housing of a scope driver, the scope driver further including:
a first rotary input driver rotatably mounted to the housing;
a drive wheel arranged within the housing and operatively coupled to the first rotary input driver such that rotation of the first rotary input driver correspondingly rotates the drive wheel;
a second rotary input driver rotatably mounted to the housing; and
an idler wheel mounted to a swing arm within the housing and operatively coupled to the second rotary input driver such that actuation of the second rotary input driver correspondingly reciprocates the idler wheel toward or away from the drive wheel;
reciprocating the swing arm of the scope driver from a first rotational position toward a second rotational position and thereby moving the idler wheel toward the drive wheel to engage the scope between the drive and idler wheels; and
rotating the drive wheel and thereby incrementally translating the scope through the channel.

20. The method of claim 19, further comprising passively rotating the idler wheel as the drive wheel rotates.
